(19) United States
(12) Patent Application Publication
(10) Pub. No.: US 2021/0246463 A1
(43) Pub. Date: Aug. 12, 2021

(54) BALANCED RESISTANCE AND AVIRULENCE GENE EXPRESSION

(71) Applicant: KWS SAAT SE & Co. KGaA, Einbeck (DE)

(72) Inventors: Dietmar Stahl, Einbeck (DE); Gabor Miklos Gyetvai, Goettingen (DE); Daniel Fabian Stirnweis, Goettingen (DE)

(21) Appl. No.: 17/253,224

(22) PCT Filed: Jun. 18, 2019
(86) PCT No.: PCT/EP2019/066096

§ 371 Date: Dec. 17, 2020

(30) Foreign Application Priority Data
Jun. 18, 2018 (EP) 18178359.8

(51) Int. Cl. C12N 15/82; C07K 14/415

(52) U.S. Cl. CPC C12N 15/8282; C12N 15/8261; C07K 14/415

(57) ABSTRACT

The invention is related to nucleic acids encoding resistance proteins, avirulence proteins, or both, wherein at least one of the proteins include at least one amino acid substitution in comparison to the wild type amino acid sequence of the resistance and/or avirulence protein, said substitution leading to a decrease of cell death during a hypersensitive reaction. Furthermore, the invention does also encompass an expression cassette, a vector, a cell, a plant, plant tissue or plant seed comprising on of the nucleic acids or one of the proteins. Finally, the invention provides a method for increasing a plant's resistance towards at least one plant pathogen.

### FIELD OF THE INVENTION

The present invention relates to a nucleic acid for increasing the resistance of a plant or a part thereof towards at least one pathogen, as well as to methods using the nucleic acid.

### BACKGROUND OF THE INVENTION

Plants possess a highly efficient, two layered innate immune system that makes them resistant against most microbial pathogens (Jones and Dangl, 2006).

The first layer of defense relies on the recognition of evolutionary conserved pathogen- or microbial-associated molecular patterns (PAMPS or MAMPs) by pattern among microbial taxa and have essential roles in microbial physiology. Only an extremely recognition receptors (PRRs). PAMPs or MAMPs are invariant structures broadly represented select group of molecules have been found to function as PAMPs (Gaudet and Gray-Owen, 2016). PRRs are generally plasma membrane receptors, which are often coupled to intracellular kinase domains or require a co-receptor to provide signaling function (Dangl et al., 2013). Depending on the presence of the signal transduction domain, the plant PRRs are classified either as receptor-like kinases (RLKs) or as receptor-like proteins (RLPs), as described by Macho and Zipfel (2014). Recognition of pathogen-associated molecular patterns (PAMPs) in the apoplast by pattern recognition receptors (PRRs) initiates a complex signaling cascade leading to PRR-triggered immunity (PTI). Adapted pathogens are able to suppress the first defense layer through the secretion of effector proteins that interfere with the signaling (Jones and Dangl, 2006, Cesari et al., 2014).

The second layer of plant defense, the effector triggered immunity (ETI), relies on the specific recognition of effectors by disease resistance genes (Jones and Dangl, 2006). This recognition leads to a strong defense response which is often associated with a local programmed cell death, the hypersensitive reaction. Since effectors are generally species- or isolate-specific, this second layer of immunity is only efficient against isolates that carry the recognized effector, which is then called an avirulence gene (Césari et al., 2014). Naturally occurring avirulence gene comprise a signaling sequence allowing for the intracellular processing within the pathogen.

The co-expression of a resistance gene and the corresponding avirulence gene in a plant cell is a promising concept to design genetically modified plants with a broad fungal resistance. The activation of resistance gene dependent defense reactions is frequently associated with cell death, the hallmark of innate immunity.

Plant resistance proteins (R-proteins) are classified into five main classes (FIG. 1), based on their structure and localization (Dangl and Jones 2001). Most resistance proteins belong to the nucleotide-binding-site leucine-rich repeat (NBS-LRR) family and are localized intracellularly. LRR domains are found in diverse proteins and function as sites of protein-protein interaction and protein-ligand binding. The recognition specificity of an NBS-LRR R-protein is usually determined by the C-terminal LRR domain. The conserved nucleotide-binding-site (NBS) domain is part of the larger NBS-ARC domain, which functions as a molecular switch which is involved in the Avr-dependent conformation change of the R-protein (Takken et al., 2006).

Depending on the N-terminal domain, NBS-LRR resistance proteins can be subdivided into CC-NBS-LRR and TIR-NBS-LRR proteins. CC-NBS-LRR proteins carry a coiled-coil (CC) domain at their N-terminus, and TIR-NBS-LRR proteins possess an N-terminus with homology to the TOLL/interleukin 1 receptor (TIR), as reviewed by Dangl and Jones, 2001. TIR-NBS-LRR proteins are completely absent from cereal species (McHale et al., 2006). The N-terminal domains are involved in downstream signaling.

The CC-domain of CC-NBS-LRR resistance proteins are classified into 2 groups according Collier and Moffet (2009) and Collier et al. (2011). The majority of CC-domains possesses a small “EDVID” consensus motif and was termed the CC_{EDVID} subtype. The EDVID motif or variations are required for the Avr dependent induction of a hypersensitive reaction as shown by amino acid substitution of the Rx protein (Rairdan et al. 2008).

The EDVID motif is absent in the CC_{RPW8} or CC_{R} domain, a less abundant subclass of CC domain. Few CC_{R}-NB-LRR proteins have been cloned to date. The best-studied members of this group include N-required gene 1 (NRG1) of Nicotiana benthamiana and activated disease resistance gene 1 (ADR1) of Arabidopsis thaliana (Collier et al., 2011). It was assumed that overexpression of an isolated CC_{EDVID} domain is not sufficient for inducing defense responses, unlike the CC_{R} domain of NRG1 or certain TIR domains (Collier et al., 2011). However, a subclass of the CC_{EDVID} domain is sufficient for cell-death induction (WO/2006/128444). This subclass is termed CC_{DAE} or CC_{DAE+EDVID} subtype, based on the presence of a typical conserved DAE motif in addition to the EDVID motif. Following this classification, the CC-NBS-LRR proteins can be divided into 3 subclasses (FIG. 1).

The functionality of a single R-gene is usually restricted to a plant family or even to a species. This phenomenon is named “restricted taxonomic functionality”. Heterologous expression of NB-LRR type R genes in a taxonomically distinct family triggers either no response or inappropriate immunity, suggesting that the regulatory or signaling components associated with NB-LRR protein based resistance are family-specific (Brutus et al., 2010). Resistance gene transfer between species may be limited not only by divergence of signaling effector molecules and pathogen avirulence ligands, but potentially also by more fundamental gene expression and transcript processing limitations (Ayliffe et al., 2004).

A break of the restricted taxonomic functionality was reported by the transfer of a pair of NB-LRR-type R-genes, RPS4 and RRS1 (Narusaka et al., 2013) from a Brassicaceae plant (Arabidopsis thaliana) into two Solanaceae plants (Nicotiana benthamiana, Solanum lycopersicum). An interfamily transfer from a Solanaceae plant (Solanum lycopersicum) to a Brassicaceae plant (Arabidopsis thaliana) was described for the RLP-type R-protein Ve1 (Fradin et al., 2011). However, although Ve1 is considered to encode a race-specific resistance protein, several observations support the hypothesis that Ve1 is an ancient pathogen receptor with traits of typical PRRs (Fradin et al., 2011). It is known that pattern recognition receptors (PRRs) can be transferred across species boundaries, as exemplified by the transfer of the PRRs EF-Tu and FLS2 from A. thaliana to N. benthamiana and S. lycopersicum. Until now, only alleles of certain single NB-LRR R-genes, as well as one two-component NB-LRR resistance gene pair were found to be suitable for an interfamily transfer from the family of Poaceae or Gramineae into the plant families of Solanaceae and Brassicaceae. The barley powdery mildew resistance gene Mla1 triggers cell death in A. thaliana after co-expression with the corresponding avirulence gene AVRₐ₁ (Lu et al., 2016). Its wheat orthologs Sr33 and Sr50 trigger cell death when overexpressed in N. benthamiana leaves (Cesari et al., 2016). The wheat powdery mildew resistance genes Pm3a and Pm3f trigger cell death after co-expression with the corresponding avirulence gene AvrPm3^{a2/f2} in N. benthamiana (Bourras et al., 2015). The avirulence gene AvrPia of Magnaporthe grisea was recognized in N. benthamiana by co-expression with the two-component NB-LRR resistance gene pair Pia_Rga4/Rga5 from rice. Generally, the distant relationship between monocot plants and dicot plants makes it difficult to predict whether a resistance gene originating from the one group is able to trigger an immune response in the other group.

The NBS-LRR resistance gene R3a of potato was cloned by a comparative genomics approach using synteny between the complex R3a locus of potato and the 12 complex locus of tomato (Huang et al., 2005). To date, R3a function was only shown for two members of the Solanaceae family (Solanum tuberosum, N. benthamiana). The Phytophthora infestans effector Avr3a, the corresponding avirulence gene of R3a, was identified with association genetics. Avr3a encodes a protein that is recognized in the host cytoplasm, where it triggers R3a-dependent cell death (Amstrong et al., 2005). The avirulence gene Avr3a occurs in P. infestans in two forms, the Avr3a_K80I103 (Avr3a^{KI}) or as Avr3a_E80M103 (Avr3a^{EM}) allele. The Avr3a^{KI} allele activates R3a resistance gene dependent innate immunity, whereas Avr3a^{EM} will not be recognized by the R3a gene (Amstrong et al. 2005). Avr3a is essential for the virulence of P. infestans and manipulates plant immunity by stabilizing potato E3 ligase CMPG1 (Bos et al., 2010). In addition, the AVR3a protein associates in potato cells with the dynamin-related protein 2 and suppresses defense responses triggered by flg22 and reduce internalization of the activated PRR FLS2 (Chaparro-Garcia et al., 2015).

An alternative approach is the expression of the avirulence gene under the control of a pathogen-inducible promoter. In the presence of a constitutively expressed R-gene, the avirulence gene should only be expressed at the time and site of pathogen infection (De Wit, 1992; Joosten and de Wit, 1999). This strategy was applied to the Cf-9/Avr9 gene pair using a 273 base-pair fragment of the pathogen-inducible promoter Gstl from potato (Martini et al., 1993). Although preliminary results showed enhanced resistance of tomato against two fungal pathogens, further optimization of the tight promoter regulation was suggested since promoter specificity was not fully restricted to the infection sites (Strittmatter at al., 1996; Joosten and de Wit, 1999).

Some transgenic plants which expressed the p50 avirulence gene under control of the 2xS-2xD pathogen-inducible promoter showed resistance to the tumor-inducing bacterium Agrobacterium tumefaciens. The bacteria resistant plants developed minor phenotypes under sterile conditions. However, after transfer to soil, all lines showed spontaneous necrosis of stems and leaves. The necrosis appeared mainly on stems and leaves of older plants. Leaf necrosis started at the tip of the leaf, spread concentrically and finally covered the whole leaf. Stem necrosis started near to soil and the attachment sites of the leaves (Niemeyer 2012).

Another scenario discussed was the dissection of cell death reaction and disease resistance (Niemeyer et al., 2013).

However, until today no agronomically attractive approach solving the underlying problem is available. Therefore, there is still a demand for plants with increased resistance or immunity (especially towards fungal pathogens) which do not show abundant cell death or necrosis.

### SUMMARY OF THE INVENTION

The present invention was made in view of the prior art described above, and the object of the present invention is to provide plants having resistance to one or more pathogens while avoiding undesired necrosis or growth retardations as well as to provide a method for the production of these plants and the agriculture application of these plants.

The present invention provides a nucleic acid molecule for increasing the resistance of a plant or a part thereof towards at least one plant pathogen, wherein the nucleic acid molecule comprises a nucleotide sequence selected from the group consisting of:

(a) a nucleotide sequence encoding i) a plant resistance protein of the resistance protein class CC-NBS-LRR and ii) an avirulence protein;

(b) a nucleotide sequence encoding a plant resistance protein of the resistance protein class CC-NBS-LRR;

(c) a nucleotide sequence encoding an avirulence protein;

(d) a nucleotide sequence hybridizing under stringent conditions to a sequence which is complementary to a nucleotide sequence according to (a), (b) or (c); and

(e) a nucleotide sequence encoding a polypeptide comprising an amino acid sequence which is at least 70% identical to an amino acid sequence according to SEQ ID NO: 93;

wherein the encoded resistance protein, the encoded avirulence protein, or both, include at least one amino acid substitution in comparison to the wild type amino acid sequence of the resistance and/or avirulence protein, said substitution leading to a decrease of cell death during a hypersensitive reaction.

A nucleic acid encoding a resistance protein as well as an avirulence protein is an artificial sequence which is manmade and which does not occur in nature. Resistance proteins are of plant origin and avirulence proteins are of plant pathogens origin. Therefore, a nucleic acid sequence encoding a resistance protein and a nucleic acid sequence encoding an avirulence protein are heterologous to each other.

A resistance protein or an avirulence protein comprising one or more amino acid substitutions as defined herein are artificial products which do not occur in nature. Also, the nucleic acids encoding these proteins are artificial and are not found in nature.

The present invention also provides a nucleic acid molecule for increasing the resistance of a plant or a part thereof towards at least one plant pathogen, wherein the nucleic acid molecule comprises a nucleotide sequence selected from the group consisting of:

(a) a nucleotide sequence encoding i) a plant resistance protein of the resistance protein class CC-NBS-LRR and ii) an avirulence protein;

(b) a nucleotide sequence encoding a plant resistance protein of the resistance protein class CC-NBS-LRR;

(c) a nucleotide sequence encoding an avirulence protein;

(d) a nucleotide sequence hybridizing under stringent conditions to a sequence which is complementary to a nucleotide sequence according to (a), (b) or (c); and

(e) a nucleotide sequence encoding a polypeptide comprising an amino acid sequence which is at least 70% identical to an amino acid sequence according to SEQ ID NO: 93;

wherein the encoded resistance protein, the encoded avirulence protein, or both, include at least one amino acid substitution wherein the substitution leads to a decrease of cell death during a hypersensitive reaction in comparison to a resistance protein, avirulence protein or both lacking the amino acid substitution. The at least one amino acid substitution can be a substitution in comparison to the wild type amino acid sequence of the resistance and/or avirulence protein.

In one embodiment, the plant resistance protein is selected from the group consisting of: R3a according to SEQ ID NO 94 as exemplarily encoded by the cDNA of SEQ ID NO 93 or the genomic DNA of SEQ ID NO 268, Rpi-blb3 according to SEQ ID NO 45 as exemplarily encoded by SEQ ID NO 44, R2 according to SEQ ID NO 49 as exemplarily encoded by SEQ ID NO 48, Rpi-abpt according to SEQ ID NO 51 as exemplarily encoded by SEQ ID NO 50, Rpi-edn1 according to SEQ ID NO 53 as exemplarily encoded by SEQ ID NO 52, R1 according to SEQ ID NO 55 as exemplarily encoded by SEQ ID NO 54, Rpi-blb2 according to SEQ ID NO 60 as exemplarily encoded by SEQ ID NO 59, Rpi-blb1 according to SEQ ID NO 65 as exemplarily encoded by SEQ ID NO 64, Rpi-pta1 according to SEQ ID NO 70 as exemplarily encoded by SEQ ID NO 69, Rpi-sto1 according to SEQ ID NO 73 as exemplarily encoded by SEQ ID NO 72, Rpi-vnt1.1 according to SEQ ID NO 76 as exemplarily encoded by SEQ ID NO 75, Rpi-edn2 according to SEQ ID NO 80 as exemplarily encoded by SEQ ID NO 79, R9a according to SEQ ID NO 266, R8 according to SEQ ID NO 86 as exemplarily encoded by SEQ ID NO 85, Rpi-chc1 according to SEQ ID NO 90 as exemplarily encoded by SEQ ID NO 89, R3b according to SEQ ID NO 98 as exemplarily encoded by SEQ ID NO 97, Mla1_barley according to SEQ ID NO 102 as exemplarily encoded by SEQ ID NO 101, Mla13_barley according to SEQ ID NO 106 as exemplarily encoded by SEQ ID NO 105, Rpg1-b_Soybean according to SEQ ID NO 111 as exemplarily encoded by SEQ ID NO 110, RPM1 according to SEQ ID NO 119 as exemplarily encoded by SEQ ID NO 118, RPS2 according to SEQ ID NO 121 as exemplarily encoded by SEQ ID NO 120, RPS5 according to SEQ ID NO 125 as exemplarily encoded by SEQ ID NO 124, BS2 according to SEQ ID NO 129 as exemplarily encoded by SEQ ID NO 128, Rxo1 according to SEQ ID NO 133 as exemplarily encoded by SEQ ID NO 132, I-2_tomato according to SEQ ID NO 137 as exemplarily encoded by SEQ ID NO 136, Pi-ta according to SEQ ID NO 144 as exemplarily encoded by SEQ ID NO 143, Piz-t according to SEQ ID NO 150 as exemplarily encoded by SEQ ID NO 149, Pia Rga4 according to SEQ ID NO 155 as exemplarily encoded by SEQ ID NO 154, Pia Rga5 according to SEQ ID NO 157 as exemplarily encoded by SEQ ID NO 156, Pii-1 according to SEQ ID NO 163 as exemplarily encoded by SEQ ID NO 162, Pik-1 according to SEQ ID NO 167 as exemplarily encoded by SEQ ID NO 166, Pik-2 according to SEQ ID NO 170 as exemplarily encoded by SEQ ID NO 169, Pikh-1 according to SEQ ID NO 173 as exemplarily encoded by SEQ ID NO 172, Pikm1-TS according to SEQ ID NO 179 as exemplarily encoded by SEQ ID NO 178, Pikm2-TS according to SEQ ID NO 182 as exemplarily encoded by SEQ ID NO 181, Pikp-1 according to SEQ ID NO 187 as exemplarily encoded by SEQ ID NO 186, Pikp-2 according to SEQ ID NO 190 as exemplarily encoded by SEQ ID NO 189, Piks-1 according to SEQ ID NO 193 as exemplarily encoded by SEQ ID NO 192, Piks-2 according to SEQ ID NO 196 as exemplarily encoded by SEQ ID NO 195, Rps1k-1_soybean according to SEQ ID NO 199 as exemplarily encoded by SEQ ID NO 198, Rps1k-2_soybean according to SEQ ID NO 205 as exemplarily encoded by SEQ ID NO 204, L3 according to SEQ ID NO 207 as exemplarily encoded by SEQ ID NO 206, N′_tobacco according to SEQ ID NO 211 as exemplarily encoded by SEQ ID NO 210, RX1 according to SEQ ID NO 213 as exemplarily encoded by SEQ ID NO 212, RX2 according to SEQ ID NO 218 as exemplarily encoded by SEQ ID NO 217, Pvr4_pepper according to SEQ ID NO 221, Pvr9_pepper according to SEQ ID NO 225 as exemplarily encoded by SEQ ID NO 224, Tm2_Tomato as exemplarily encoded by SEQ ID NO 228 as exemplarily encoded by SEQ ID NO 227, Tm2\hat{ }2_Tomato according to SEQ ID NO 232 as exemplarily encoded by SEQ ID NO 231, Tsw_pepper according to SEQ ID NO 234 as exemplarily encoded by SEQ ID NO 233, Sw-5b_tomato according to SEQ ID NO 238 as exemplarily encoded by SEQ ID NO 237, Rsv1_3gG2 according to SEQ ID NO 242 as exemplarily encoded by SEQ ID NO 241, Pm2 according to SEQ ID NO 246 as exemplarily encoded by SEQ ID NO 245, PM3A according to SEQ ID NO 252 as exemplarily encoded by SEQ ID NO 251, and PM3F according to SEQ ID NO 258 as exemplarily encoded by SEQ ID NO 257.

According to one embodiment, the resistance protein is not the potato Rx resistance protein or a mutated form thereof and/or not the tomato Cf-9 resistance protein or a mutated form thereof.

In a specific embodiment, the nucleic acid according to the invention is encoding a resistance protein, wherein the nucleic acid molecule encodes a plant resistance protein having at least one amino acid substitution within the CC-domain in comparison to the amino acid sequence of a wild type CC-domain. Alternatively, the encoded plant resistance protein can include at least one amino acid substitution within the CC-domain wherein the substitution leads to a decrease of cell death during a hypersensitive reaction in comparison to a resistance protein, lacking the amino acid substitution within the CC-domain.

According to a further aspect of the invention the amino acid substitution(s) within the resistance protein or the avirulence protein or both of them is not a substitution/are not substitutions that result(s) in a protein variant which is existing in nature. Therefore, a substitution turning the naturally occurring Avr3a-KI protein (including the signal sequence) to the naturally occurring Avr3a-KM protein (including the signal sequence) or the other way round would be excluded according to this aspect.

In one embodiment, the NBS domain of the resistance protein may comprise an NBS motif.

In one embodiment, the LRR domain of the resistance protein may comprise an Leu-xx-Leu-xx-Leu-x-Leu-xx-Cys/Asn-xx motif according to SEQ ID NO 272 (where x is any amino acid).

In another embodiment, the resistance protein is a resistance protein which comprises the amino acid motif according to SEQ ID NO 273 as depicted in FIG. 3.

In one embodiment, the avirulence protein is selected from the group consisting of: Avr3a_Phytophthora according to SEQ ID NO 96 as exemplarily encoded by SEQ ID NO 95, Avr2_Phytophthora_infestans according to SEQ ID NO 139 as exemplarily encoded by SEQ ID NO 138, Avr1_Phytophthora_infestans according to SEQ ID NO 58 as exemplarily encoded by SEQ ID NO 57, Avrblb2 according to SEQ ID NO 63 as exemplarily encoded by SEQ ID NO 62, Avr-blb1=Avr-sto1 according to SEQ ID NO 68 as exemplarily encoded by SEQ ID NO 67, Avr-vnt1 according to SEQ ID NO 78 as exemplarily encoded by SEQ ID NO 77, Avrblb2 according to SEQ ID NO 82 as exemplarily encoded by SEQ ID NO 81, Avr8_Phytophthora_infestans according to SEQ ID NO 88 as exemplarily encoded by SEQ ID NO 87, PITG_16245 according to SEQ ID NO 92 as exemplarily encoded by SEQ ID NO 91, Avr3b according to SEQ ID NO 100 as exemplarily encoded by SEQ ID NO 99, Avra1_(AvrMla1)_Blumeria according to SEQ ID NO 104 as exemplarily encoded by SEQ ID NO 103, Avra13_(AvrMla13)_Blumeria according to SEQ ID NO 109 as exemplarily encoded by SEQ ID NO 108, AvrB_Pseudomonas according to SEQ ID NO 113 as exemplarily encoded by SEQ ID NO 112, AvrRpm1 according to SEQ ID NO 115 as exemplarily encoded by SEQ ID NO 114, AvrPpiA1_Pseudomonas according to SEQ ID NO 117 as exemplarily encoded by SEQ ID NO 116, AvrRpt2_Pseudomonas according to SEQ ID NO 123 as exemplarily encoded by SEQ ID NO 122, AvrPphB according to SEQ ID NO 127 as exemplarily encoded by SEQ ID NO 126, AvrBs2_Xanthomonas according to SEQ ID NO 131 as exemplarily encoded by SEQ ID NO 130, AvrRxo1 according to SEQ ID NO 135 as exemplarily encoded by SEQ ID NO 134, Avr2 (SIX3)_Fusarium oxysporum according to SEQ ID NO 139 as exemplarily encoded by SEQ ID NO 138, SIX5_Fusarium oxysporum according to SEQ ID NO 141 as exemplarily encoded by SEQ ID NO 140, AVR Pita according to SEQ ID NO 147 as exemplarily encoded by SEQ ID NO 146, AvrPiz-t according to SEQ ID NO 153 as exemplarily encoded by SEQ ID NO 152, AVR1-C039 according to SEQ ID NO 159 as exemplarily encoded by SEQ ID NO 158, AVR-Pia according to SEQ ID NO 161 as exemplarily encoded by SEQ ID NO 160, Avr-Pii according to SEQ ID NO 165 as exemplarily encoded by SEQ ID NO 164, Avr Pik_km_kp allele D according to SEQ ID NO 185 as exemplarily encoded by SEQ ID NO 184, Avr1k_Phytophthora sojae according to SEQ ID NO 201 as exemplarily encoded by SEQ ID NO 200, CP_Pepper mild mottle virus according to SEQ ID NO 209 as exemplarily encoded by SEQ ID NO 208, CP_Potato virus X according to SEQ ID NO 216 as exemplarily encoded by SEQ ID NO 215, Nlb_pepper mottle virus according to SEQ ID NO 223 as exemplarily encoded by SEQ ID NO 222, 30 kDa MP_Tobacco mosaic virus according to SEQ ID NO 230 as exemplarily encoded by SEQ ID NO 229, NSs_Tomato spotted wilt virus (AvrTsw) according to SEQ ID NO 236 as exemplarily encoded by SEQ ID NO 235, NSm_Tomato spotted wilt virus according to SEQ ID NO 240 as exemplarily encoded by SEQ ID NO 239, P3 cistron_soybean mosaic virus according to SEQ ID NO 244 as exemplarily encoded by SEQ ID NO 243, AvrPm2 according to SEQ ID NO 249 as exemplarily encoded by SEQ ID NO 248, and AvrPm3a2_f2 according to SEQ ID NO 255 as exemplarily encoded by SEQ ID NO 254 wherein preferably at least one of the amino acids of such a protein is substituted.

In a preferred embodiment, the nucleic acid molecule comprises a nucleotide sequence encoding a plant resistance protein of the resistance protein class CC-NBS-LRR and an avirulence protein, wherein the plant resistance protein and the avirulence protein are a combination selected from the following combinations: R3a (CC; AAW48299) and Avr3a_Phytophthora, Rpi-blb3 and Avr2_Phytophthora_infestans, R2 and Avr2_Phytophthora_infestans, Rpi-abpt and Avr2_Phytophthora_infestans, pi-edn1 and Avr2_Phytophthora_infestans, R1 and Avr1_Phytophthora_infestans, Rpi-blb2 and Avrblb2, Rpi-blb1 and Avr-blb1=Avr-sto1, Rpi-pta1 and Avr-blb1=Avr-sto1, Rpi-sto1 and Avr-blb1=Avr-sto1, Rpi-vnt1.1 and Avr-vnt1 (PITG_16294)_Phytophthora, Rpi-edn2 and Avrblb2, R9a and Avrblb2, R8 and Avr8_Phytophthora_infestans, Rpi-chc1 and PITG_16245 (Avr-chc1)_Phytophthora, R3b (CC; AEC47890) and Avr3b (PITG_18215)_Phytophthora, Mla1_barley and Avra1_(AvrMla1)_Blumeria, Mla13_barley and Avra13_(AvrMla13)_Blumeria, Rpg1-b_Soybean and AvrB_Pseudomonas, RPM1 (CC; CAA61131) and AvrB_Pseudomonas, RPM1 (CC; CAA61131) and AvrRpm1, RPM1 (CC; CAA61131) and AvrPpiA1_Pseudomonas, RPS2 (CC; AEE85156) and AvrRpt2_Pseudomonas, RPS5 (CC; AEE28852) and AvrPphB (HopAR1)_Pseudomonas, BS2 (CC; AAF09256) and AvrBs2_Xanthomonas, Rxo1 (AAX31149) and AvrRxo1, 1-2_tomato and Avr2 (SIX3)_Fusarium oxysporum, 1-2_tomato and SIX5_Fusarium oxysporum, Pi-ta (AF207842) and AVR Pita (AF207841), Piz-t (ABC73398) and AvrPiz-t (ACF39937), Pia Rga4 (BAK39922) and AVR1-C039 (AF463528_3), Pia Rga4 (BAK39922) and AVR-Pia (BAH59484), Pia Rga5 (BAK39930) and AVR1-C039 (AF463528_3), Pia Rga5 (BAK39930) and AVR-Pia (BAH59484), Pii-1 (BAN59294) and Avr-Pii (BAH59485), Pik-1 (ADZ48537) and Avr Pik_km_kp allele D (allele pex31-D) (BAH59486), Pik-2 (ADZ48538) and Avr Pik_km_kp allele D (allele pex31-D) (BAH59486), Pikh-1 (AET36549) and Avr Pik_km_kp allele D (allele pex31-D) (BAH59486), Pikh-2 (AET36550) and Avr Pik_km_kp allele D (allele pex31-D) (BAH59486), Pikm1-TS (BAG71909) and Avr Pik_km_kp allele D (allele pex31-D) (BAH59486), Pikm2-TS (BAG71908) and Avr Pik_km_kp allele D (allele pex31-D) (BAH59486), Pikp-1 (ADV58352) and Avr Pik_km_kp allele D (allele pex31-D) (BAH59486), Pikp-2 (ADV58351) and Avr Pik_km_kp allele D (allele pex31-D) (BAH59486), Piks-1 (AET36547) and Avr Pik_km_kp allele D (allele pex31-D) (BAH59486), Piks-2 (AET36548) and Avr Pik_km_kp allele D (allele pex31-D) (BAH59486), Rps1k-1_soybean and Avr1k_Phytophthora sojae, Rps1k-2_soybean and Avr1k_Phytophthora sojae, L3 (CC; BAJ33559) and CP_Pepper mild mottle virus (Avr L3&4), N′_tobacco and CP_Pepper mild mottle virus (Avr L3&4), RX1 (CC; CAB50786) and CP_Potato virus X, RX2 (CC; CAB56299) and CP_Potato virus X, Pvr4_pepper and Nlb_pepper mottle virus (Avr Pvr4&9), Pvr9_pepper and Nlb_pepper mottle virus (Avr Pvr4&9), Tm2_Tomato and 30 kDa MP_Tobacco mosaic virus (AvrTm2), Tm2\hat{ }2_Tomato and 30 kDa MP_Tobacco mosaic virus (AvrTm2), Tsw_pepper and NSs_Tomato spotted wilt virus (AvrTsw), Sw-5b_tomato and NSm_Tomato spotted wilt virus (AvrSw-5b), Rsv1_3gG2 and P3 cistron_soybean mosaic virus (AvrRsv1_3gG2), Pm2 and AvrPm2, PM3A (CC; AAY21626) and AvrPm3a2_f2, and PM3F and AvrPm3a2_f2.

In one embodiment, the nucleic acid molecule comprises a nucleotide sequence encoding the avirulence protein Avr3a lacking the protein signal sequence and having at least one amino acid substitution in the amino acid sequence of SEQ ID NO: 2, wherein the at least one amino acid substitution is selected from the group consisting of: K59/M82, R59/M82, H59/M82, C59/M82, N59/M82, Q59/M82, T59/M82, S59/M82, M59/M82, G59/M82, A59/M82, L59/M82, V59/M82, 159/M82. In this respect an amino acid substitution “R59/M82” means that the amino acid at position 59 is replaced by the amino acid arginine (“R”) and the amino acid at position 82 is replaced by methionine (“M”). In this respect the positions 59 and 82 can also correspond to the respective position in a variant of the protein according to SEQ ID NO 2. An example for a sequence encoding a protein according to SEQ ID NO 2 is given by the nucleic acid molecule according to SEQ ID NO 1. Therefore, the following artificial sequences of Avr3a having amino acid substitutions are part of the invention:

| | SEQ ID NO | |
|---|---|---|
| | Nucleic acid sequence | SEQ ID NO |
| | encoding the protein | Protein sequence |
| K59/I82 | 1 | 2 |
| K59/M82 | 4 | 5 |
| R59/M82 | 6 | 7 |
| H59/M82 | 8 | 9 |
| D59/M82 | 10 | 11 |
| E59/M82 | 12 | 13 |
| F59/M82 | 14 | 15 |
| Y59/M82 | 16 | 17 |
| W59/M82 | 18 | 19 |
| P59/M82 | 20 | 21 |
| C59/M82 | 22 | 23 |
| N59/M82 | 24 | 25 |
| Q59/M82 | 26 | 27 |
| T59/M82 | 28 | 29 |
| S59/M82 | 30 | 31 |
| M59/M82 | 32 | 33 |
| G59/M82 | 34 | 35 |
| A59/M82 | 36 | 37 |
| L59/M82 | 38 | 39 |
| V59/M82 | 40 | 41 |
| I59/M82 | 42 | 43 |

Avirulence proteins which occur in nature are proteins that are secreted (usually from the pathogen to the infested plant) and therefore naturally occurring avirulence proteins have a signal sequence. Avirulence proteins which lack the signal sequence and the nucleic acids which encode such proteins are manmade and do not occur in nature.

In one embodiment, the nucleic acid molecule comprises a nucleotide sequence encoding the wildtype avirulence protein Avr3a including the signal sequence and having at least one amino acid substitution in the amino acid sequence of SEQ ID NO: 267, wherein the at least one amino acid substitution is selected from the group consisting of: K80/I103 (already included in SEQ ID NO 267), K80/M103, R80/M103, H80/M103, C80/M103, N80/M103, Q80/M103, T80/M103, S80/M103, M80/M103, G80/M103, A80/M103, L80/M103, V80/M103, I80/M103. An example for a sequence encoding a protein according to SEQ ID NO 267 is given by the nucleic acid molecule according to SEQ ID NO 3.

In one embodiment, the nucleic acid molecule comprises a nucleotide sequence encoding a plant resistance protein having at least one amino acid substitution within the CC-domain of the wild type protein. Preferably, the at least one amino acid substitution in the plant resistance protein is selected from the group consisting of: (a) an amino acid substitution within the DAE motif of resistance protein BvKWS3_165 encoded by the nucleic acid sequence according to SEQ ID NO 274 wherein the DAE motif expands from amino acid position 56 to amino acid position 58 in the amino acid sequence according to SEQ ID NO 263,; (b) an amino acid substitution within the DAE motif of resistance protein Bv123 encoded by the nucleic acid sequence according to SEQ ID NO 276 wherein the DAE motif expands from amino acid position 57 to amino acid position 59 of SEQ ID NO: 261, and (c) an amino acid substitution within the DAE motif of resistance protein R3a wherein the DAE motif expands from amino acid position 59 to amino acid position 61 of SEQ ID NO: 265. The amino acid sequence of SEQ ID NO: 263 is exemplarily encoded by the nucleic acid molecule according to SEQ ID NO: 262. More preferably, the at least one amino acid substitution is within the D or E of the DAE motif of BvKWS3_165, Bv123, or R3a. The amino acid sequence of SEQ ID NO: 261 is exemplarily encoded by the nucleic acid molecule according to SEQ ID NO: 260. The amino acid sequence of SEQ ID NO: 265 is exemplarily encoded by the nucleic acid molecule according to SEQ ID NO: 264.

In a preferred embodiment, the plant resistance protein is R3a having the wild type amino acid sequence of SEQ ID NO: 269, wherein said wild type amino acid sequence has at least one substitution selected from the group consisting of: K35E, K37M, E61V, E61K, E135N und E136N. In the case of “K35E” this means for example that the amino acid K at position 35 is replaced by E.

Moreover, provided is a cell comprising the nucleic acid molecule, the polypeptide, or the vector or expression cassette of the present invention. Preferably, the cell comprises the nucleic acid molecule of the present invention as a transgene or as an endogene. The cell may be for example an eucaryotic cell like a plant cell or a yeast cell. However, the cell may be for example also a procaryotic cell like a bacterial cell wherein the bacterial cell may be for example a cell of E. coli or Agrobacterium tumefaciens.

Further provided is a plant or part thereof comprising the nucleic acid molecule of the present invention or comprising the cell of the present invention, and a plant or part thereof regenerated from said cell.

Also provided is a seed or offspring from the plant of the present invention, wherein the seed or the offspring comprises the nucleic acid molecule of the present invention as a transgene or an endogene.

The present invention also provides a method for increasing a plant's resistance towards at least one plant pathogen. In one embodiment, said method comprises the steps of integrating the nucleic acid molecule of the present invention into the genome of at least one cell of a plant and regenerating a plant from that cell. Alternatively, said method comprises the steps of transforming a plant cell with a nucleic acid molecule of the present invention, or the vector or the expression cassette of the present invention, and regenerating a plant from that cell. Further, the present invention provides a method for the identification of a plant comprising the nucleic acid molecule of the present invention, said method comprising the steps of isolating DNA from the plant of the present invention or part thereof, and performing a polymerase chain reaction using the isolated DNA as a template, thereby amplifying the nucleic acid molecule of the present invention or a part thereof. Preferably the method for identification will detect not only the presence of a nucleic acid molecule according to the present invention but will also detect at least one of the amino acid substitutions encoded by the nucleic acid molecule.

### BRIEF DESCRIPTION OF THE DRAWINGS

FIG. 1: Classification of plant resistance genes into five groups (modified after Dangl and Jones, 2001). TIR-NBS-LRR and CC-NBS-LRR proteins are located in the cytoplasm of the plant cells. They have a NBS (nucleotide binding site) and a LRR (leucine rich repeat) domain in common. They differ at the N-terminus by the TIR (Toll/interleukin receptor) and CC (coiled-coil) domain. The CC-NBS-LRR will be subdivided into the CC_{EDVID}-NBS-LRR, CC_{DAE}-NBS-LRR and CC_{R}-NBS-LRR types according this patent disclosure. RLK (receptor-like-kinase) and RLP (receptor-like-proteins) are membrane-bound receptors. A RLK receptor has a LRR at the N-terminus, followed by a transmembrane domain and a kinase domain at the C-terminus. The RLP receptor consists of a LRR domain, followed by a transmembrane domain. The Pto like kinase type consists of a kinase domain.

FIG. 2: Detrimental effect of R3a-Avr3a co-expression in adult but not in young plants. The transgenic lines T-047, T-036, T-029 and T-071, which were transformed with the R3a-Avr3a genes, showed leaf abortion and stunted growth as adult but not as young plants. Expression analysis by qRT-PCR showed that the Avr3a gene is stronger expressed in young plants than in adult plants. The negative phenotype of adult R3a-Avr3a lines correlates with Avr3a^{KI} expression. Avr3a^{KI} expression was normalized against expression of the potato house-keeping gene StMCB75. n=4, ±indicates standard derivation.

FIG. 3: Comparison of the CC_{DAE}-domains from the R-proteins BvKWS3_165 according to SEQ ID NO 263 exemplarily encoded by the nucleic acid according to SEQ ID NO 262, Bv123 protein according to SEQ ID NO 261 exemplarily encoded by the nucleic acid according to SEQ ID NO 260 and StR3a protein according to SEQ ID NO 265 exemplarily encoded by the nucleic acid according to SEQ ID NO 264. Identical amino acids of the resistance proteins from sugar beet (BvKWS3_165, Bv123) and potato (StR3a) are highlighted with black background. The DAE motif, which was used for the classification of the CC-type, is highly conserved and located at position 56-58 (BvKWS3_165), 57-59 (Bv123) and 59-61 (StR3a). The EDVID motif is conserved as EDILD consensus sequence and located at position 82-86 (BvKWS3_165), 83-87 (Bv123) and 85-89 (StR3a). The size of the CC-domains is given as numbers, e.g. #175=175 amino acids. Also depicted is a consensus motif according to SEQ ID NO 273 deduced from the three sequences.

FIG. 4a and FIG. 4b: Generation of R3a gene variants with reduced cell death activity by single amino acid exchanges of the CC_{DAE}-domain (FIG. 4a). Generated R3a alleles showed reduced cell death after co-expression with the Avr3a^{KI} gene. In each construct (the last six columns), one amino acid of the CC_{DAE}-domain was exchanged in the CC-NBS-LRR protein (e.g. K35E=K at position 35 was replaced by E). The strongest cell death was observed for the native R3a gene (3rd column showing results for “AVR3a+R3a”). The number describe the level of cell death after ballistic transformation of potato leaves (0=max. cell death, 100=no cell death detected). pCaMV-2=empty vector (negative control). The standard derivation of the mean from 3 experiments with 6 replicates is given by error bars (FIG. 4b).

FIG. 5 to FIG. 8: Allelic variants of the Avr3a gene trigger different levels of cell death in potato leaves. Avr3a variants under the control of the doubled 35S promoter trigger different levels of cell death after transient expression in potato leaves of the R3a cultivar Hermes. The mean of three independent experiments is shown (n=18). Empty vector (expression vector without Avr3a gene) was set to 100, Avr3aKI (native avirulent allele). The Shapiro Wilk test was performed for normality distribution of the data. Data with different letters are significantly different according ANOVA and Tukey test (multiple t-test, p<0.05).

FIG. 9: Comparison of cell death strength induced by allelic variants of the Avr3a gene (s. FIG. 5-8) in potato leaves.

FIG. 10: Phenotype of the potato lines Avr3a^{GM}-T-024 and Avr3a^{IM}-T-112 compared to the potato cultivar Hermes. Adult plants of lines transformed with the Avr3a^{GM} and Avr3a^{IM} gene under the control of the synthetic-fungal inducible promoter 2xS-4xD-NpCABE showed the same phenotype as the cultivar Hermes, which was used for transformation.

FIG. 11a and FIG. 11b: Enhanced late blight resistance of line Avr3a^{GM}-T-024 in comparison to Hermes and a transgenic control (RNAi-GFP). The top 5 leaflets of each potato leaf were locally inoculated with one drop of sporangia (20 μl with 600 sporangia). Photos were taken at 5 dpi.

FIG. 12a and FIG. 12b: Expression analysis of the Avr3a gene in P. infestans infected and control leaves of potato by qRT-PCR. The transgenic line with the best late blight resistance, Avr3a^{GM}-T-024, showed the highest expression of the recombinant Avr3a gene after infection at 2, 3 and 4 dpi. Selected primers S3169 and S3170 allowed the specific detection of the recombinant Avr3a gene expression in the presence of P. infestans. Avr3a expression was normalized against the potato gene StMCB75. Black column=infected leaves, grey column=control leaves. n=4. dpi=days after inoculation, ko=non-infected leaves. ±indicates standard derivation.

FIG. 13: The threshold of cell death induction is increased by application of Avr3a^{GM} in comparison to the native Avr3a^{KI} gene. Expression of the Avr3a^{KI} and Avr3a^{GM} genes in leaves of adult greenhouse plants was determined by qRT-PCR using the gene specific primers S3169 and S3170. The transgenic lines T-047, T-036, T-029 and T-071, which were transformed with the R3a-Avr3a^{KI} genes, showed leaf abortion and stunted growth (see FIG. 2). The level of leaf damage correlated with the Avr3a^{KI} expression. Even the Avr3a^{KI} low expressing line T-047 showed visible leaf abortion. This indicated that the threshold for cell death induction is less than 1.8 relative units for the Avr3a^{KI} expressing lines. In contrast, the R3a-Avr3a^{GM} co-expressing line Avr3a^{GM}-T024 did not show a negative phenotype and revealed a Avr3a^{GM} expression of 146.5±30.1 relative units. This result demonstrated that the threshold for cell death induction is greater than 200 relative units for the modified Avr3a gene in line Avr3a^{GM}-T-024. Avr3a expression was normalized against expression of the potato house-keeping gene StMCB75 n=4, ±indicates standard derivation.

FIG. 14a and FIG. 14b: Enhanced early blight (Alternaria solani) resistance of line Avr3a^{GM}-T-024 in comparison to Hermes and a transgenic control (RNAi-GFP). Greenhouse plants were scored for natural A. solani infection (n=8 rows with 3 plants each). 0=healthy leaves, 100=completely destroyed leaves. Differences between samples were assessed by using analysis of variance (ANOVA). *Significant different according ANOVA to Hermes, RNAi-GFP and Avr3a^{GM}-T-014, p<0.05.

FIG. 15a, FIG. 15b and FIG. 15c: R3a-Avr3a^{KI} co-expression activates cell death, the hallmark of innate immunity, in corn, soybean and wheat leaves. The potato R3a resistance gene and the Avr3a^{KI} gene of P. infestans were transiently co-expressed in leaves of corn, soybean and wheat. The induction of cell death was measured by the activity of two co-bombarded luciferase reporter genes 16 h after transformation. An auto-activated wheat resistance gene and an auto-activated soybean resistance gene were used as “positive controls” for wheat, corn and soybean.

FIG. 16a, FIG. 16b and FIG. 16c: In contrast to R3a-Avr3a^{KI} co-expression, the co-expression of R1-Avr1 does not activate cell death in corn, soybean and wheat leaves. The potato R1 resistance gene and the Avr1 gene of P. infestans were transiently co-expressed in leaves of corn, soybean and wheat. The induction of cell death was measured by the activity of two co-bombarded luciferase reporter genes 16 h after transformation; an auto-activated wheat resistance gene and an auto-activated soybean resistance gene were used as “positive controls” for wheat, corn and soybean.

FIG. 17a, FIG. 17b and FIG. 17c: In contrast to R3a-Avr3a^{KI} co-expression, the co-expression of Rx1-PVX-CP does not activate cell death in corn, soybean and wheat leaves. The potato Rx1 resistance gene and the gene encoding the coat protein of Potato virus X (PVX-CP) were transiently co-expressed in leaves of corn, soybean and wheat. The induction of cell death was measured by the activity of two co-bombarded luciferase reporter genes 16 h after transformation. An auto-activated wheat resistance gene and an auto-activated soybean resistance gene were used as “positive controls” for wheat, corn and soybean.

FIG. 18: Vectormap of the vector pBIN. The vector comprises the AVR3a gene as avirulence gene and R3a as resistance gene. The avirulence gene is under control of a promoter construct comprising the NpCABE minimal promoter and the 2xS-4xD cis-regulatory elements. The resistance gene is under control of the R3a promoter. The genes NPTII, NPTIII and tetR are included in the vector for conferring resistance to selective agents. LB=left border of the T-DNA (transfer DNA); RB=right border of the T-DNA; the vector is suitable for stable integration of the avirulence gene and the resistance gene into a plant genome and subsequent expression of the genes.

FIG. 19: Schematic construction of an expression cassette including the resistance gene R3a and the avirulence gene Avr3a^{KI}. The expression cassette is usable for expression of the included genes.

### DETAILED DESCRIPTION OF THE INVENTION

The present invention is based on the discovery that genetically modified genes involved in the gene-for-gene interaction can be used to decrease cell death and to prevent the undesired activation of defense reactions in the absence of pathogens. Mutagenized versions of native resistance genes or Avr genes having reduced cell death-inducing activity were generated and applied, in order to decrease cell death, e.g. by increasing the threshold for the induction of cell death. Genetic elements of the gene-for-gene interaction having reduced cell death-inducing activity were shown to decrease cell death and to prevent the undesired activation of defense reactions in the absence of pathogens. At the same time, the modified genes were still capable of activating the innate immunity during pathogen infection. The inventive technology was successfully tested for the CC-NBS-LRR resistance gene R3a of potato and the Avr3a gene of P. infestans. The same technology may be applied to any CC-NBS-LRR resistance gene of a crop and the matching avirulence gene. In addition, it was shown that the R3a-Avr3a gene combination induces cell death in corn, soybean and wheat. Hence, the findings of the present invention were successfully transferred to these other crops. A list of additional CC-NBS-LRR resistance genes and their corresponding AVR genes which may be used in the methods of the invention is provided.

Accordingly, the present invention provides a nucleic acid molecule for increasing the resistance of a plant or a part thereof towards at least one plant pathogen, wherein the nucleic acid molecule comprises a nucleotide sequence selected from the group consisting of:

(a) a nucleotide sequence encoding i) a plant resistance protein of the resistance protein class CC-NBS-LRR and ii) an avirulence protein;

(b) a nucleotide sequence encoding a plant resistance protein of the resistance protein class CC-NBS-LRR;

(c) a nucleotide sequence encoding an avirulence protein;

(d) a nucleotide sequence hybridizing under stringent conditions to a sequence which is complementary to a nucleotide sequence according to (a), (b) or (c); and

(e) a nucleotide sequence encoding a polypeptide comprising an amino acid sequence which is at least 70% identical to an amino acid sequence according to SEQ ID NO: 93 or 271;

wherein the encoded resistance protein, the encoded avirulence protein, or both, include at least one amino acid substitution in comparison to the wild type amino acid sequence of the resistance and/or avirulence protein, said substitution leading to a decrease of cell death during a hypersensitive reaction.

Preferably, the at least one amino acid substitution is an amino acid substitution outside of the signal sequence of the encoded resistance protein or the encoded avirulence protein.

The expression “resistance” or “resistant” as regards a pathogen should be understood to mean the ability of a plant or plant cell to resist the damaging effects of the pathogen and extends from a delay in the development of disease to complete suppression of the development of the disease. The resistance may be complete or partial and may be specific or non-specific to the pathogen race. A conferred resistance may be a newly inherited resistance or an increase in a partial resistance which is already extant.

Resistance may be quantified by methods known in the art. For example, resistance may be quantified by a reduced fungal biomass on the host plant; for this, the fungal DNA may be determined with the aid of quantitative PCR in comparison to the plant DNA in the infested plant tissue. An additional approach to the measurement of resistance is optical rating, wherein rating scores of 1 (not susceptible) to 9 (very susceptible) are awarded. An increased resistance can be measured by for example inoculation of a healthy leaf with an isolate of the pathogen and the determination of the infested surface after determined number of days (for example 15 days). A reduce of 5% of the infested surface corresponds to an increase of the resistance of 5%.

Plant: A plant can be a dicot or a monocot. Plants which can be agronomically exploited are preferred. Examples of plants which can be used in context of the invention are sugar beet or red beet; potato; corn; cotton, rape; Poaceae including cereals like wheat, barley, rye, rice, oat, millet; sugar cane, sunflower; Fabaceae like soybean, beans, peanuts, lentils and lupins.

Part of a plant: In general, every organ or tissue of a plant form a part of a plant. A part of a plant can be leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, and cuttings.

Pathogen: A “pathogen” as used herein refers to an organism which can infect a plant, or which can cause a disease in a plant. Pathogens which can infect a plant, or which can cause a disease in a plant, include fungi, oomycetes, bacteria, viruses, viroids, virus-like organisms, phytoplasmas, protozoa, nematodes and parasitic plants. Plant parasites can cause damage by feeding on a plant and can be selected from ectoparasites like insects, comprising aphids and other sap-sucking insect, mites, and vertebrates. Examples for fungal pathogens which are especially suitable to be combated by the present invention are Alternaria solani, Phytophthora infestans, Blumeria graminis or fungi belonging to the genus of Fusarium.

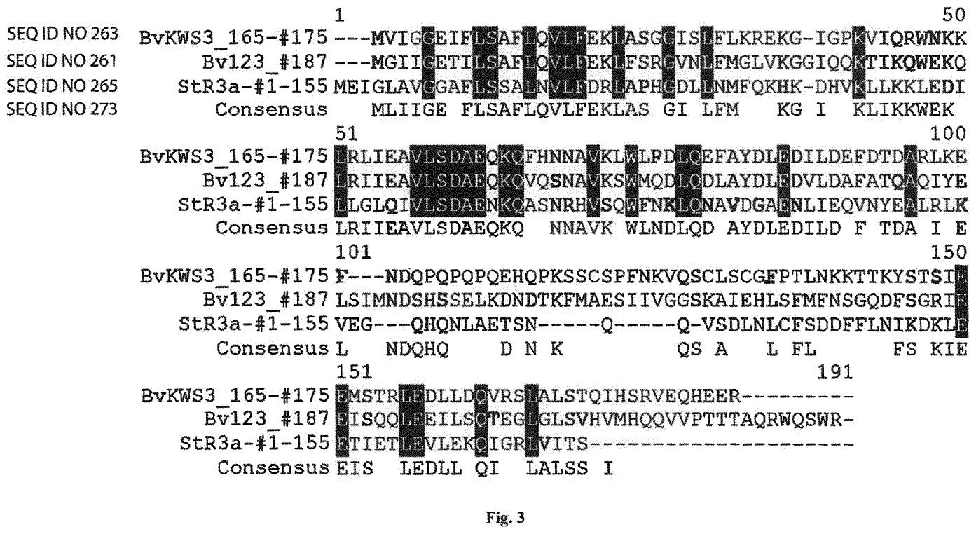

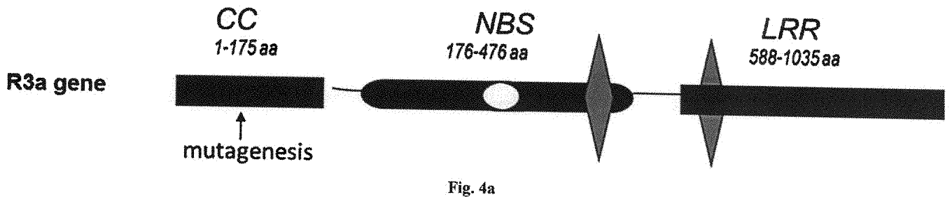

Plant resistance protein: The plant resistance proteins are key molecules for the activation of the induced defense mechanisms. According to the gene-for-gene postulate a resistance interacts directly or indirectly with a corresponding protein encoded by pathogen's avirulence gene (Avr-gene) and thereby triggers the induced defensive reaction. CC-NBS-LRR proteins may comprise three domains: A coiled-coil domain (CC), a NBS comain (nucleotide binding site) and a LRR domain (leucin-rich repeat). In the context of this invention CC-NBS-LRR proteins may also be such proteins which comprise the amino acid sequence VLSDAE or the motif VLSDAEXKQ as depticted in FIG. 3 (wherein X can be every genetically encodable amino acid). This motif is also suitable to characterize or identify the CC domain wherein the cc domain my beginn 56 amino acids in n-terminal direction from the position of the amino acid sequence VLSDAE according to SEQ ID NO: 282 or the motif VLSDAEXKQ (with V being position 0) according to SEQ ID NO: 283. Furthermore, the CC domain may stretch 129 amino acids towards in c-terminal direction from the position of the amino acid sequence according to SEQ ID NO: 282 (with E being position 0) or 127 amino acids from the position of the motif according to SEQ ID NO: 283 (with Q being position 0).

Avirulence protein: An avirulence protein is produced by a plant pathogen and is able for direct or indirect chemical interaction with a corresponding plant resistance protein wherein this interaction triggers a hypersensitive reaction (defense reaction) within a plant cell.

Variant: Variants of proteins or of nucleic acids encompass for example homologs, orthologs, or allelic variants.

Hybridize: The term “hybridize” or “hybridization” should be understood to mean a procedure in which a single stranded nucleic acid molecule agglomerates with a nucleic acid strand which is as complementary as possible, i.e. base-pairs with it. Examples of standard methods for hybridization have been described in 2001 by Sambrook et al. Preferably, this should be understood to mean that at least 60%, more preferably at least 65%, 70%, 75%, 80% or 85%, particularly preferably 90%, 91%, 92%, 93%, 94%, 95%, 96%, 97%, 98% or 99% of the bases of the nucleic acid molecule undergo base pairing with the nucleic acid strand which is as complementary as possible. The possibility of such agglomeration depends on the stringency of the hybridization conditions. The term “stringency” refers to the hybridization conditions. High stringency is when base pairing is more difficult, low stringency is when base pairing is easier.

The stringency of the hybridization conditions depends, for example, on the salt concentration or ionic strength and the temperature. In general, the stringency can be increased by raising the temperature and/or by reducing the salt content. The term “stringent hybridization conditions” should be understood to mean those conditions under which a hybridization takes place primarily only between homologous nucleic acid molecules. The term “hybridization conditions” in this respect refers not only to the actual conditions prevailing during actual agglomeration of the nucleic acids, but also to the conditions prevailing during the subsequent washing steps. Examples of high stringent hybridization conditions are conditions under which primarily only those nucleic acid molecules that have at least 90% or at least 95% sequence identity undergo hybridization. Such high stringent hybridization conditions are, for example: 4×SSC at 65° C. and subsequent multiple washes in 0.1×SSC at 65° C. for approximately 1 hour. The term “high stringent hybridization conditions” as used herein may also mean: hybridization at 68° C. in 0.25 M sodium phosphate, pH 7.2, 7% SDS, 1 mM EDTA and 1% BSA for 16 hours and subsequently washing twice with 2×SSC and 0.1% SDS at 68° C. Preferably, hybridization takes place under stringent conditions. Less stringent hybridization conditions are, for example: hybridizing in 4×SSC at 37° C. and subsequent multiple washing in 1×SSC at room temperature.

Hypersensitive reaction: A plant defense reaction including the controlled cellular death of the host tissue at the infection site, the strengthening of the plant cell wall by lignification and callose formation, the formation of phytoalexins and the production of PR-(pathogenesis-related) proteins.

Decrease/reduction of cell death and reduced cell death-inducing activity: Characteristic of a protein which comprises at least one amino acid substitution and is suitable to increase the resistance towards at least one pathogen. The decrease or reduction of cell death is evident due to a reduced number of leaf abortion during the development of a plant in comparison to a plant comprising the protein having the corresponding wild type amino acid sequence without substitution of at least one amino acid.

An alternative approach to proof that a resistance inducing protein leads to decreased or reduced cell death is the biolistic transformation of a leaf with an expression cassette comprising a nucleic acid encoding a protein according to the invention and a second leaf treated in the same way using a sequence encoding a protein without amino acid substitution wherein both leaves are co-transformed with a luciferase reporter gene. After 16 h the induction of cell death is measured by the activity of the expressed luciferase. A nucleic acid leading to more luciferase activity than the nucleic acid of the comparison-leaf shows reduced induction of cell death.

Signal sequence of a protein: An amino acid sequence within a protein which is usually located in the n-terminal part of a protein and can for example begin after the first n-terminal amino acid. Signal sequences usually consist of 13-36 amino acids. In most cases they possess a central, strong hydrophobic part which extends over 10-15 amino acids along which often can be found: alanine, leucine, valine, isoleucine and phenylalanine. The signal sequence is usually responsible for the transportation of the protein to its final location within the cell. However, the signal sequence is not always necessary for the function of a protein and is often removed after the protein has reached its final localization. For example, a plant's immune system usually can recognize pathogenic Avr proteins even if those Avr proteins lack their naturally occurring signal sequence.

Wild type amino acid sequence or wild type protein: A sequence of amino acids building a protein compound which is existing in nature.

Substitution leading to a decrease of cell death during a hypersensitive reaction: Is a substitution in the amino acid sequence of a resistance protein and/or avirulence protein. The cell death rate during or as consequence of a hypersensitive reaction (wherein the triggering or maintenance of the hypersensitive reaction is affected either by the resistance protein or the avirulence protein or both) is reduced in comparison to a hypersensitive reaction (occurring under comparable circumstances) the triggering or maintenance of which is affected by the resistance protein or avirulence protein or both lacking the substitution. However, the proteins comprising the substitution are still able to affect the triggerance or maintenance of a hypersensitive reaction and are therefore functional proteins wherein substitution leading to a complete loss of function are not encompassed.

In one embodiment, the plant resistance protein is selected from the group consisting of: R3a (CC; AAW48299), Rpi-blb3, R2, Rpi-abpt, Rpi-edn1, R1, Rpi-blb2, Rpi-blb1, Rpi-pta1, Rpi-sto1, Rpi-vnt1.1, Rpi-edn2, R9a, R8, Rpi-chc1, R3b (CC; AEC47890), Mla1_barley, Mla13_barley, Rpg1-b_Soybean, RPM1 (CC; CAA61131), RPS2 (CC; AEE85156), RPS5 (CC; AEE28852), BS2 (CC; AAF09256), Rxo1 (AAX31149), I-2_tomato, Pi-ta (AF207842), Piz-t (ABC73398), Pia Rga4 (BAK39922), Pia Rga5 (BAK39930), Pii-1 (BAN59294), Pik-1 (ADZ48537), Pik-2 (ADZ48538), Pikh-1 (AET36549), Pikm1-TS (BAG71909), Pikm2-TS (BAG71908), Pikp-1 (ADV58352), Pikp-2 (ADV58351), Piks-1 (AET36547), Piks-2 (AET36548), Rps1k-1_soybean, Rps1k-2_soybean, L3 (CC; BAJ33559), N′_tobacco, RX1 (CC; CAB50786), RX2 (CC; CAB56299), Pvr4_pepper, Pvr9_pepper, Tm2_Tomato, Tm2\hat{ }2_Tomato, Tsw_pepper, Sw-5b_tomato, Rsv1_3gG2, Pm2, PM3A (CC; AAY21626), and PM3F.

In another embodiment, the avirulence protein is selected from the group consisting of: Avr3a_Phytophthora, Avr2_Phytophthora_infestans, Avr1_Phytophthora_infestans, Avrblb2, Avr-blb1=Avr-sto1, Avr-vnt1 (PITG_16294)_Phytophthora, Avrblb2, Avr8_Phytophthora_infestans, PITG_16245 (Avr-chc1)_Phytophthora, Avr3b (PITG_18215)_Phytophthora, Avra1_(AvrMla1)_Blumeria, Avra13_(AvrMla13)_Blumeria, AvrB_Pseudomonas, AvrRpm1, AvrPpiA1_Pseudomonas, AvrRpt2_Pseudomonas, AvrPphB (HopAR1)_Pseudomonas, AvrBs2_Xanthomonas, AvrRxo1, Avr2 (SIX3)_Fusarium oxysporum, SIX5_Fusarium oxysporum, AVR Pita (AF207841), AvrPiz-t (ACF39937), AVR1-C039 (AF463528_3), AVR-Pia (BAH59484), Avr-Pii (BAH59485), Avr Pik_km_kp allele D (allele pex31-D) (BAH59486), Avr1 k_Phytophthora sojae, CP_Pepper mild mottle virus (Avr L3&4), CP_Potato virus X, Nlb_pepper mottle virus (Avr Pvr4&9), 30 kDa MP_Tobacco mosaic virus (AvrTm2), NSs_Tomato spotted wilt virus (AvrTsw), NSm_Tomato spotted wilt virus (AvrSw-5b), P3 cistron_soybean mosaic virus (AvrRsv1_3gG2), AvrPm2, and AvrPm3a2_f2.

In yet another embodiment, the nucleic acid molecule comprises a nucleotide sequence encoding a plant resistance protein of the resistance protein class CC-NBS-LRR and an avirulence protein, wherein the plant resistance protein and the avirulence protein are a combination selected from the following combinations: R3a (CC; AAW48299) and Avr3a_Phytophthora, Rpi-blb3 and Avr2_Phytophthora_infestans, R2 and Avr2_Phytophthora_infestans, Rpi-abpt and Avr2_Phytophthora_infestans, pi-edn1 and Avr2_Phytophthora_infestans, R1 and Avr1_Phytophthora_infestans, Rpi-blb2 and Avrblb2, Rpi-blb1 and Avr-blb1=Avr-sto1, Rpi-pta1 and Avr-blb1=Avr-sto1, Rpi-sto1 and Avr-blb1=Avr-sto1, Rpi-vnt1.1 and Avr-vnt1 (PITG_16294)_Phytophthora, Rpi-edn2 and Avrblb2, R9a and Avrblb2, R8 and Avr8_Phytophthora_infestans, Rpi-chc1 and PITG_16245 (Avr-chc1)_Phytophthora, R3b (CC; AEC47890) and Avr3b (PITG_18215)_Phytophthora, Mla1_barley and Avra1_(AvrMla1)_Blumeria, Mla13_barley and Avra13_(AvrMla13)_Blumeria, Rpg1-b_Soybean and AvrB_Pseudomonas, RPM1 (CC; CAA61131) and AvrB_Pseudomonas, RPM1 (CC; CAA61131) and AvrRpm1, RPM1 (CC; CAA61131) and AvrPpiA1_Pseudomonas, RPS2 (CC; AEE85156) and AvrRpt2_Pseudomonas, RPS5 (CC; AEE28852) and AvrPphB (HopAR1)_Pseudomonas, BS2 (CC; AAF09256) and AvrBs2_Xanthomonas, Rxo1 (AAX31149) and AvrRxo1, 1-2_tomato and Avr2 (SIX3)_Fusarium oxysporum, 1-2_tomato and SIX5_Fusarium oxysporum, Pi-ta (AF207842) and AVR Pita (AF207841), Piz-t (ABC73398) and AvrPiz-t (ACF39937), Pia Rga4 (BAK39922) and AVR1-C039 (AF463528_3), Pia Rga4 (BAK39922) and AVR-Pia (BAH59484), Pia Rga5 (BAK39930) and AVR1-C039 (AF463528_3), Pia Rga5 (BAK39930) and AVR-Pia (BAH59484), Pii-1 (BAN59294) and Avr-Pii (BAH59485), Pik-1 (ADZ48537) and Avr Pik_km_kp allele D (allele pex31-D) (BAH59486), Pik-2 (ADZ48538) and Avr Pik_km_kp allele D (allele pex31-D) (BAH59486), Pikh-1 (AET36549) and Avr Pik_km_kp allele D (allele pex31-D) (BAH59486), Pikh-2 (AET36550) and Avr Pik_km_kp allele D (allele pex31-D) (BAH59486), Pikm1-TS (BAG71909) and Avr Pik_km_kp allele D (allele pex31-D) (BAH59486), Pikm2-TS (BAG71908) and Avr Pik_km_kp allele D (allele pex31-D) (BAH59486), Pikp-1 (ADV58352) and Avr Pik_km_kp allele D (allele pex31-D) (BAH59486), Pikp-2 (ADV58351) and Avr Pik_km_kp allele D (allele pex31-D) (BAH59486), Piks-1 (AET36547) and Avr Pik_km_kp allele D (allele pex31-D) (BAH59486), Piks-2 (AET36548) and Avr Pik_km_kp allele D (allele pex31-D) (BAH59486), Rps1k-1_soybean and Avr1 k_Phytophthora sojae, Rps1k-2_soybean and Avr1 k_Phytophthora sojae, L3 (CC; BAJ33559) and CP_Pepper mild mottle virus (Avr L3&4), N′_tobacco and CP_Pepper mild mottle virus (Avr L3&4), RX1 (CC; CAB50786) and CP_Potato virus X, RX2 (CC; CAB56299) and CP_Potato virus X, Pvr4_pepper and Nlb_pepper mottle virus (Avr Pvr4&9), Pvr9_pepper and Nlb_pepper mottle virus (Avr Pvr4&9), Tm2_Tomato and 30 kDa MP_Tobacco mosaic virus (AvrTm2), Tm2\hat{ }2_Tomato and 30 kDa MP_Tobacco mosaic virus (AvrTm2), Tsw_pepper and NSs_Tomato spotted wilt virus (AvrTsw), Sw-5b_tomato and NSm_Tomato spotted wilt virus (AvrSw-5b), Rsv1_3gG2 and P3 cistron_soybean mosaic virus (AvrRsv1_3gG2), Pm2 and AvrPm2, PM3A (CC; AAY21626) and AvrPm3a2_f2, and PM3F and AvrPm3a2_f2.

In one embodiment the nucleic acid molecule according to the invention encodes
i) a plant resistance protein of the resistance protein class CC-NBS-LRR and
ii) an avirulence protein;
and wherein the plant resistance protein and the avirulence protein are a combination selected from the group consisting of:
i) SEQ ID NO 94 and SEQ ID NO 96,
ii) SEQ ID NO 45 and SEQ ID NO 47,
iii) SEQ ID NO 49 and SEQ ID NO 47,
iv) SEQ ID NO 51 and SEQ ID NO 47,
v) SEQ ID NO 53 and SEQ ID NO 47,
vi) SEQ ID NO 55 and SEQ ID NO 58,
vii) SEQ ID NO 60 and SEQ ID NO 63,
viii) SEQ ID NO 65 and SEQ ID NO 68,
ix) SEQ ID NO 70 and SEQ ID NO 68,
x) SEQ ID NO 73 and SEQ ID NO 68,
xi) SEQ ID NO 76 and SEQ ID NO 78,
xii) SEQ ID NO 80 and SEQ ID NO 82,
xiii) SEQ ID NO 266 and SEQ ID NO 82,
xiv) SEQ ID NO 86 and SEQ ID NO 88,
xv) SEQ ID NO 90 and SEQ ID NO 92,
xvi) SEQ ID NO 98 and SEQ ID NO 100,
xvii) SEQ ID NO 102 and SEQ ID NO 104,
xviii) SEQ ID NO 106 and SEQ ID NO 109,
xix) SEQ ID NO 111 and SEQ ID NO 113,
xx) SEQ ID NO 119 and SEQ ID NO 113,
xxi) SEQ ID NO 119 and SEQ ID NO 115,
xxii) SEQ ID NO 119 and SEQ ID NO 117,
xxiii) SEQ ID NO 121 and SEQ ID NO 123,
xxiv) SEQ ID NO 125 and SEQ ID NO 127,
xxv) SEQ ID NO 129 and SEQ ID NO 131,
xxvi) SEQ ID NO 133 and SEQ ID NO 135,
xxvii) SEQ ID NO 137 and SEQ ID NO 139,
xxviii) SEQ ID NO 137 and SEQ ID NO 141,
xxix) SEQ ID NO 144 and SEQ ID NO 147,
xxx) SEQ ID NO 150 and SEQ ID NO 153,
xxxi) SEQ ID NO 155 and SEQ ID NO 159,
xxxii) SEQ ID NO 155 and SEQ ID NO 161,
xxxiii) SEQ ID NO 157 and SEQ ID NO 159,
xxxiv) SEQ ID NO 157 and SEQ ID NO 161,
xxxv) SEQ ID NO 163 and SEQ ID NO 165,
xxxvi) SEQ ID NO 167 and SEQ ID NO 185,
xxxvii) SEQ ID NO 176 and SEQ ID NO 185,
xxxviii) SEQ ID NO 173 and SEQ ID NO 185,
xxxix) SEQ ID NO 176 and SEQ ID NO 185,
xl) SEQ ID NO 179 and SEQ ID NO 185,
xli) SEQ ID NO 182 and SEQ ID NO 185,
xlii) SEQ ID NO 187 and SEQ ID NO 185,
xliii) SEQ ID NO 190 and SEQ ID NO 185,
xliv) SEQ ID NO 193 and SEQ ID NO 185,
xlv) SEQ ID NO 196 and SEQ ID NO 185,
xlvi) SEQ ID NO 199 and SEQ ID NO 201,
xlvii) SEQ ID NO 205 and SEQ ID NO 201,
xlviii) SEQ ID NO 207 and SEQ ID NO 209,
xlix) SEQ ID NO 211 and SEQ ID NO 209,
l) SEQ ID NO 213 and SEQ ID NO 216,
li) SEQ ID NO 218 and SEQ ID NO 216,
lii) SEQ ID NO 221 and SEQ ID NO 223,
liii) SEQ ID NO 225 and SEQ ID NO 223,
liv) SEQ ID NO 228 and SEQ ID NO 230,
lv) SEQ ID NO 232 and SEQ ID NO 230,
lvi) SEQ ID NO 234 and SEQ ID NO 236,
lvii) SEQ ID NO 238 and SEQ ID NO 240,
lviii) SEQ ID NO 242 and SEQ ID NO 244,
lix) SEQ ID NO 246 and SEQ ID NO 249,
lx) SEQ ID NO 252 and SEQ ID NO 255, and
lxi) SEQ ID NO 258 and SEQ ID NO 255.

Preferably, the combination of genes is R3a (CC; AAW48299) and Avr3a_Phytophthora.

The following table gives further information about CC-NBS-LRR resistance genes from different crops and their corresponding Avr genes suitable to be used in the invention wherein the use as a combination of resistance gene and corresponding Avr gene is preferred: Table 1: List of CC-NBS-LRR resistance proteins with matching avirulence proteins.

TABLE 1
List of CC-NBS-LRR resistance proteins with matching avirulence proteins.

| Resistance proteins: | corresponding Avirulence proteins | | |
|---|---|---|---|
| Rpi-blb3 | Avr2_Phytophthora_infestans | | |
| SEQ ID NO 44 | SEQ ID NO 46 | | |
| (SEQ ID NO 45) | (SEQ ID NO 47) | | |
| R2 | Avr2_Phytophthora_infestans | | |
| SEQ ID NO 48 | SEQ ID NO 46 | | |
| (SEQ ID NO 49) | (SEQ ID NO 47) | | |
| Rpi-abpt | Avr2_Phytophthora_infestans | | |
| SEQ ID NO 50 | SEQ ID NO 46 | | |
| (SEQ ID NO 51) | (SEQ ID NO 47) | | |
| Rpi-edn1 | Avr2_Phytophthora_infestans | | |
| SEQ ID NO 52 | SEQ ID NO 46 | | |
| (SEQ ID NO 53) | (SEQ ID NO 47) | | |
| R1 | Avr1_Phytophthora_infestans | | |
| SEQ ID NO 54 | SEQ ID NO 57 | | |
| (SEQ ID NO 55) | (SEQ ID NO 58) | | |
| [SEQ ID NO 56] | | | |
| Rpi-blb2 | Avrblb2 | | |
| SEQ ID NO 59 | SEQ ID NO 62 | | |
| (SEQ ID NO 60) | (SEQ ID NO 63) | | |
| [SEQ ID NO 61] | | | |
| Rpi-blb1 | Avr-blb1 = Avr-sto1 | | |
| SEQ ID NO 64 | SEQ ID NO 67 | | |
| (SEQ ID NO 65) | (SEQ ID NO 68) | | |
| [SEQ ID NO 66] | | | |
| Rpi-pta1 | Avr-blb1 = Avr-sto1 | | |
| SEQ ID NO 69 | SEQ ID NO 67 | | |
| (SEQ ID NO 70) | (SEQ ID NO 68) | | |
| [SEQ ID NO 71] | | | |
| Rpi-sto1 | Avr-blb1 = Avr-sto1 | | |
| SEQ ID NO 72 | SEQ ID NO 67 | | |
| (SEQ ID NO 73) | (SEQ ID NO 68) | | |
| [SEQ ID NO 74] | | | |
| Rpi-vnt1.1 | Avr-vnt1 | | |
| SEQ ID NO 75 | (PITG_16294)_Phytophthora | | |
| (SEQ ID NO 76) | SEQ ID NO 77 | | |
| | (SEQ ID NO 78) | | |
| Rpi-edn2 | Avrblb2 | | |
| SEQ ID NO 79 | SEQ ID NO 81 | | |
| (SEQ ID NO 80) | (SEQ ID NO 82) | | |
| R9a | Avrblb2 | | |
| (SEQ ID NO 266) | SEQ ID NO 81 | | |
| | (SEQ ID NO 82) | | |
| R8 | Avr8_Phytophthora_infestans | | |
| SEQ ID NO 85 | SEQ ID NO 87 | | |
| (SEQ ID NO 86) | (SEQ ID NO 88) | | |
| Rpi-chc1 | PITG_16245 (Avr- | | |
| SEQ ID NO 89 | chc1)_Phytophthora | | |
| (SEQ ID NO 90) | SEQ ID NO 91 | | |
| | (SEQ ID NO 92) | | |
| R3a (CC; AAW48299) | Avr3a_Phytophthora | | |
| SEQ ID NO 93 | SEQ ID NO 95 | | |
| (SEQ ID NO 94) | (SEQ ID NO 96) | | |
| R3b (CC; AEC47890) | Avr3b | | |
| SEQ ID NO 97 | (PITG_18215)_Phytophthora | | |
| (SEQ ID NO 98) | SEQ ID NO 99 | | |
| | (SEQ ID NO 100) | | |
| Mla1_barley | Avra1_(AvrMla1)_Blumeria | | |
| SEQ ID NO 101 | SEQ ID NO 103 | | |
| (SEQ ID NO 102) | (SEQ ID NO 104) | | |
| Mla13_barley | Avra13_(AvrMla13)_Blumeria | | |
| SEQ ID NO 105 | SEQ ID NO 108 | | |
| (SEQ ID NO 106) | (SEQ ID NO 109) | | |
| [SEQ ID NO 107] | | | |
| Rpg1-b_Soybean | AvrB_Pseudomonas | | |
| SEQ ID NO 110 | SEQ ID NO 112 | | |
| (SEQ ID NO 111) | (SEQ ID NO 113) | | |
| RPM1 (CC; | | AvrRpm1 | AvrPpiA1_Pseudomonas |
| CAA61131) | | SEQ ID NO 114 | SEQ ID NO 116 |
| SEQ ID NO 118 | | (SEQ ID NO 115) | (SEQ ID NO 117) |
| (SEQ ID NO 119) | | | |
| RPS2 (CC; | AvrRpt2_Pseudomonas | | |
| AEE85156) | SEQ ID NO 122 | | |
| SEQ ID NO 120 | (SEQ ID NO 123) | | |
| (SEQ ID NO 121) | | | |
| RPS5 (CC; | AvrPphB (HopAR1)_Pseudomonas | | |
| AEE28852) | SEQ ID NO 126 | | |
| SEQ ID NO 124 | (SEQ ID NO 127) | | |
| (SEQ ID NO 125) | | | |
| B52 (CC; AAF09256) | AvrBs2_Xanthomonas | | |
| SEQ ID NO 128 | SEQ ID NO 130 | | |
| (SEQ ID NO 129) | (SEQ ID NO 131) | | |
| Rxo1 (AAX31149) | AvrRxo1 | | |
| SEQ ID NO 132 | SEQ ID NO 134 | | |
| (SEQ ID NO 133) | (SEQ ID NO 135) | | |
| I-2_tomato | Avr2 (SIX3)_Fusarium oxysporum | SIX5_Fusarium | |
| SEQ ID NO 136 | SEQ ID NO 138 | oxysporum | |
| (SEQ ID NO 137) | (SEQ ID NO 139) | SEQ ID NO 140 | |
| | | (SEQ ID NO 141) | |
| | | [SEQ ID NO 142] | |
| Pi-ta (AF207842) | AVR Pita (AF207841) | | |
| SEQ ID NO 143 | SEQ ID NO 146 | | |
| (SEQ ID NO 144) | (SEQ ID NO 147) | | |
| [SEQ ID NO 145] | [SEQ ID NO 148] | | |
| Piz-t (ABC73398) | AvrPiz-t (ACF39937) | | |
| SEQ ID NO 149 | SEQ ID NO 152 | | |
| (SEQ ID NO 150) | (SEQ ID NO 153) | | |
| (SEQ ID NO 151) | | | |
| Pia Rga4 (BAK39922) | AVR1-CO39 (AF463528_3) | | |
| SEQ ID NO 154 | SEQ ID NO 158 | | |
| (SEQ ID NO 155) | (SEQ ID NO 159) | | |
| Pia Rga5 (BAK39930) | AVR-Pia (BAH59484) | | |
| SEQ ID NO 156 | SEQ ID NO 160 | | |
| (SEQ ID NO 157) | (SEQ ID NO 161) | | |
| Pii-1 (BAN59294) | Avr-Pii (BAH59485) | | |
| SEQ ID NO 162 | SEQ ID NO 164 | | |
| (SEQ ID NO 163) | (SEQ ID NO 165) | | |
| Pik-1 (ADZ48537) | Avr Pik_km_kp allele D (allele | | |
| SEQ ID NO 166 | pex31-D) (BAH59486) | | |
| (SEQ ID NO 167) | SEQ ID NO 184 | | |
| Pik-2 (ADZ48538) | (SEQ ID NO 185) | | |
| SEQ ID NO 169 | Avr Pik_km_kp allele D (allele | | |
| (SEQ ID NO 170) | pex31-D) (BAH59486) | | |
| [SEQ ID NO 171] | SEQ ID NO 184 | | |
| Pikh-1 (AET36549) | (SEQ ID NO 185) | | |
| SEQ ID NO 172 | | | |
| (SEQ ID NO 173) | | | |
| [SEQ ID NO 174] | | | |
| Pikh-2 (AET36550) | | | |
| SEQ ID NO 175 | | | |
| (SEQ ID NO 176) | | | |
| [SEQ ID NO 177] | | | |
| Pikm1-TS | | | |
| (BAG71909) | | | |
| SEQ ID NO 178 | | | |
| (SEQ ID NO 179) | | | |
| [SEQ ID NO 180] | | | |
| Pikm2-TS | | | |
| (BAG71908) | | | |
| SEQ ID NO 181 | | | |
| (SEQ ID NO 182) | | | |
| [SEQ ID NO 183] | | | |
| Pikp-1 (ADV58352) | | | |
| SEQ ID NO 186 | | | |
| (SEQ ID NO 187) | | | |
| [SEQ ID NO 188] | | | |
| Pikp-2 (ADV58351) | | | |
| SEQ ID NO 189 | | | |
| (SEQ ID NO 190) | | | |
| [SEQ ID NO 191] | | | |
| Piks-1 (AET36547) | | | |
| SEQ ID NO 192 | | | |
| (SEQ ID NO 193) | | | |
| [SEQ ID NO 194] | | | |
| Piks-2 (AET36548) | | | |
| SEQ ID NO 195 | | | |
| (SEQ ID NO 196) | | | |
| [SEQ ID NO 197] | | | |
| Rps1k-l_soybean | Avr1k_Phytophthora sojae | Avr1b_Phyto- | |
| SEQ ID NO 198 | SEQ ID NO 200 | phthora sojae | |
| (SEQ ID NO 199) | (SEQ ID NO 201) | SEQ ID NO 202 | |
| Rps1k-2_soybean | | (SEQ ID NO 203) | |
| SEQ ID NO 204 | | | |
| (SEQ ID NO 205) | | | |
| L3 (CC; BAJ33559) | CP_Pepper mild mottle virus | | |
| SEQ ID NO 206 | (Avr L3&4) | | |
| (SEQ ID NO 207) | SEQ ID NO 208 | | |
| | (SEQ ID NO 209) | | |
| N′tobacco | CP_Pepper mild mottle virus | | |
| SEQ ID NO 210 | (Avr L3&4) | | |
| (SEQ ID NO 211) | SEQ ID NO 208 | | |
| | (SEQ ID NO 209) | | |
| RX1 (CC; CAB50786) | CP_Potato virus X | | |
| SEQ ID NO 212 | SEQ ID NO 215 | | |
| (SEQ ID NO 213) | (SEQ ID NO 216) | | |
| [SEQ ID NO 214] | | | |
| RX2 (CC; CAB56299) | | | |
| SEQ ID NO 217 | | | |
| (SEQ ID NO 218) | | | |
| [SEQ ID NO 219] | | | |
| Pvr4_pepper | Nlb_pepper mottle virus | | |
| SEQ ID NO 220 | (Avr Pvr4&9) | | |
| (SEQ ID NO 221) | SEQ ID NO 222 | | |
| Pvr9_pepper | (SEQ ID NO 223) | | |
| SEQ ID NO 224 | | | |
| (SEQ ID NO 225) | | | |
| [SEQ ID NO 226] | | | |
| Tm2_Tomato | 30kDa MP_Tobacco mosaic virus | | |
| SEQ ID NO 227 | (AvrTm2) | | |
| (SEQ ID NO 228) | SEQ ID NO 229 | | |
| Tm2\hat{ }2_Tomato | (SEQ ID NO 230) | | |
| SEQ ID NO 231 | | | |
| (SEQ ID NO 232) | | | |
| Tsw_pepper | NSs_Tomato spotted wilt virus | | |
| SEQ ID NO 233 | (AvrTsw) | | |
| (SEQ ID NO 234) | SEQ ID NO 235 | | |
| | (SEQ ID NO 236) | | |
| Sw-5b_tomato | NSm_Tomato spotted wilt virus | | |
| SEQ ID NO 237 | (AvrSw-5b) | | |
| (SEQ ID NO 238) | SEQ ID NO 239 | | |
| | (SEQ ID NO 240) | | |
| Rsv1_3gG2 | P3 cistron_soybean mosaic virus- | | |
| SEQ ID NO 241 | AvrRsv1_ 3gG2 | | |
| (SEQ ID NO 242) | SEQ ID NO 243 | | |
| | (SEQ ID NO 244) | | |
| Pm2 | AvrPm2 | | |
| SEQ ID NO 245 | SEQ ID NO 248 | | |
| (SEQ ID NO 246) | (SEQ ID NO 249) | | |
| [SEQ ID NO 247] | [SEQ ID NO 250] | | |
| PM3A (CC; | AvrPm3a2_f2 | | |
| AAY21626) | SEQ ID NO 254 | | |
| SEQ ID NO 251 | (SEQ ID NO 255) | | |
| (SEQ ID NO 252) | [SEQ ID NO 256] | | |
| [SEQ ID NO 253] | | | |
| PM3F | | | |
| SEQ ID NO 257 | | | |
| (SEQ ID NO 258) | | | |
| [SEQ ID NO 259] | | | |

Detailed sequence information is given in an additional listing. Not all known functional protein variants encoded by different alleles are listed. SEQ ID NO=nucleotide sequence as cDNA; (SEQ ID NO in curved brackets=amino acid sequence); [SEQ ID NO in square brackets=genomic sequence]

As is apparent from Tab. 1 for example the combination of resistance protein RPM1 (or the gene encoding the protein) with one of the avirulence proteins AvrB_Pseudomonas or AvrRpm1 or AvrPpiA1_Pseudomonas (or with the genes encoding these proteins) within a plant cell can trigger (directly or indirectly) a hyper sensitive immune response. Each of the genes can be modified as described elsewhere herein to reduce or decrease the decrease the cell death during the hypersensitive reaction.

The plant resistance protein and/or the avirulence protein of the present invention comprises at least one amino acid substitution compared to the respective wild type amino acid sequence, resulting in a reduction or decrease of cell death during a hypersensitive reaction.

In one embodiment the nucleic acid molecule according to the invention encodes an avirulence protein comprising at least one amino acid substitution selected from the group consisting of: K59/M82, R59/M82, H59/M82, C59/M82, N59/M82, Q59/M82, T59/M82, S59/M82, M59/M82, G59/M82, A59/M82, L59/M82, V59/M82, I59/M82 within the amino acid sequence of SEQ ID NO: 2. This means that the nucleic acid molecule encodes an avirulence protein comprising essentially the amino acid according to SEQ ID NO: 2 but that this sequence is modified by one or more of the above given amino acid substitutions. This avirulence protein may be the avirulence protein Avr3a wherein the avirulence protein optionally lacks a signal sequence.

It is also possible to use the native avirulence protein Avr3a including the signal sequence and substituting amino acids according to the following table:

TABLE 2
Avr3a variants comprising different substitutions according to the invention

| | SEQ ID NO | | |
|---|---|---|---|
| | Nucleic acid | | |
| | sequence | SEQ ID NO | Chemical property |
| | encoding the | Protein | of amino acid at |
| | protein | sequence | position 59 |
| K59/I82 | 1 | 2 | basic |
| K59/M82 | 4 | 5 | basic |
| R59/M82 | 6 | 7 | basic |
| H59/M82 | 8 | 9 | basic |
| D59/M82 | 10 | 11 | acidic |
| E59/M82 | 12 | 13 | acidic |
| F59/M82 | 14 | 15 | aromatic |
| Y59/M82 | 16 | 17 | aromatic |
| W59/M82 | 18 | 19 | aromatic |
| P59/M82 | 20 | 21 | non-polar |
| C59/M82 | 22 | 23 | polar |
| N59/M82 | 24 | 25 | polar |
| Q59/M82 | 26 | 27 | polar |
| T59/M82 | 28 | 29 | polar |
| S59/M82 | 30 | 31 | polar |
| M59/M82 | 32 | 33 | non-polar |
| G59/M82 | 34 | 35 | non-polar |
| A59/M82 | 36 | 37 | non-polar |
| L59/M82 | 38 | 39 | aliphatic |
| V59/M82 | 40 | 41 | aliphatic |
| I59/M82 | 42 | 43 | aliphatic |

Twenty Avr3aE59X/M82 variants and the native Avr3aK59/I82 allele are listed together with the corresponding substitution positions and SEQ ID NOs. Furthermore, the chemical property of the amino acid at position 59 is given.

In one embodiment the nucleic acid molecule according to the invention encodes a resistance protein comprising at least one amino acid substitution selected from the group consisting of: K35E, K37M, E61V, E61K, E135N and E136N within the amino acid sequence of SEQ ID NO: 269. This means that the nucleic acid molecule encodes a plant resistance protein comprising essentially the amino acid according to SEQ ID NO: 269 but that this sequence is modified by one or more of the above given amino acid substitutions. This resistance protein may be R3a.

Further provided is a polypeptide encoded by the nucleic acid molecule of the present invention. The polypeptide may be a plant resistance protein or an avirulence protein. The avirulence protein may be lacking the naturally occurring signal sequence which is responsible for the intracellular processing of the avirulence protein in the cell of the pathogen. An avirulence protein lacking the signal sequence is a protein that does not occur in nature. The invention does also encompass a combination of at least one resistance protein and at least one avirulence protein wherein at least one of the proteins comprises at least one amino acid substitution as described elsewhere herein. Potential combinations of resistance genes and avirulence genes are disclosed elsewhere herein. The invention does also encompass a solution comprising at least one protein or at least one combination of proteins according to the invention. The solution may be an aqueous solution and may comprise further compounds like a pH buffer and stabilizers. Part of the invention is also a method for the exterior or interior application of the solution to a plant or a plant part. The method comprises the steps of providing a plant or plant part and application of the solution to the outer or inner tissue of the plant or its part. The solution may be sprayed or injected.

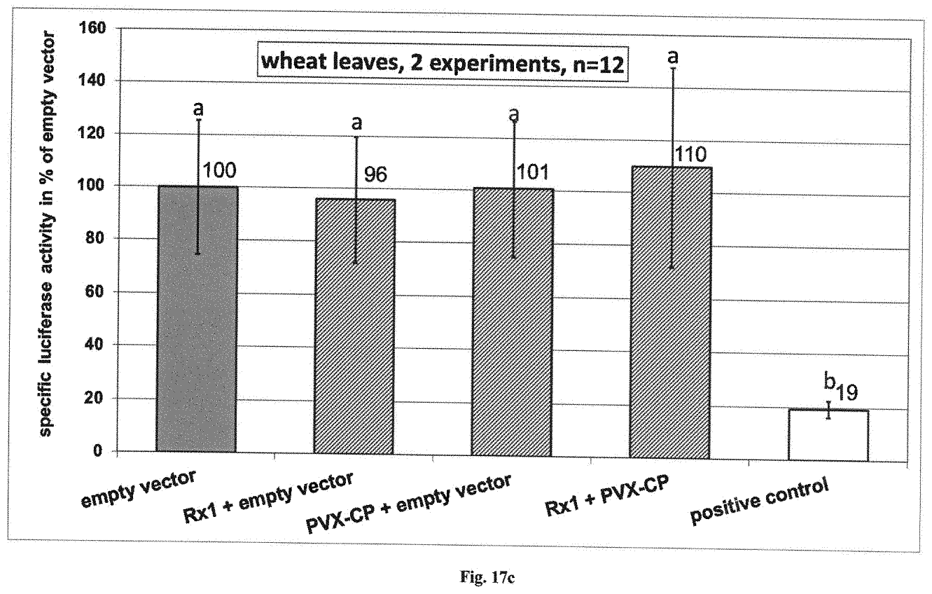

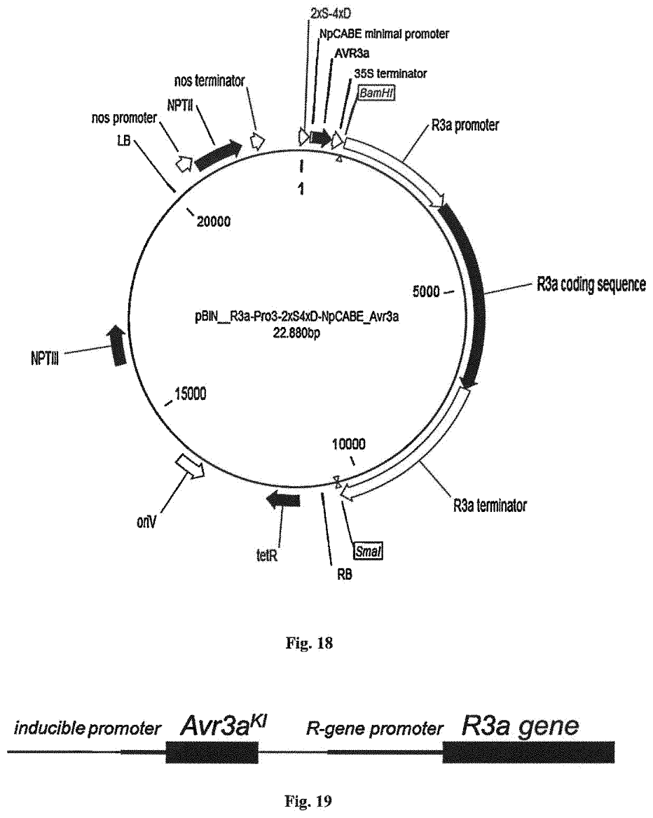

Also provided is a vector or expression cassette comprising the nucleic acid molecule of the present invention. An example of a vector according to the invention is illustrated by FIG. 18. The illustrated vector includes coding sequences for the proteins R3a and Avr3a. The structure of these two proteins can be adapted by including amino acid substitutions as described herein. An example of an expression cassette is given by SEQ ID NO 270. Furthermore FIG. 19 gives an illustration of a suitable expression cassette.

The nucleic acid according to the invention encoding a resistance protein or an avirulence protein or both may be under control of a pathogen-inducible promoter or a promoter heterologous to the resistance protein or the avirulence protein or both. The pathogen-inducible promoter may be a synthetic pathogen-inducible promoter. Synthetic pathogen-inducible promoters are manmade and do not occur in nature.

The vector may be a plasmid, a cosmid, a phage or an expression vector, a transformation vector, shuttle vector or cloning vector, it may be double or single stranded, linear or circular, or it may be a prokaryotic or eukaryotic host, either by integration into its genome or transforming extrachromosomally. The vector may be heterologous to the resistance protein or the avirulence protein or both. Vectors are synthetic constructs which are manmade and which to not occur in nature. Preferably, the nucleotide sequence of the invention is operably linked in an expression vector to one or more regulatory sequences which allow transcription and optionally expression in a prokaryotic or eukaryotic host cell. As an example, the nucleotide sequence may be under the control of a suitable promoter or a terminator. Suitable promoters may be promoters which are constitutively induced (see, for example, the 35S promoter from the “cauliflower mosaic virus” (Odell et al. 1985)); particularly suitable promoters are those promoters which are pathogen-inducible (see, for example, the PR1 promoter from parsley (Rushton et al., 1996)). Particularly suitable pathogen-inducible promoters are synthetic or chimeric promoters which do not occur in nature, are composed of several elements and contain a minimum promoter as well as, upstream of the minimum promoter, at least one cis-regulatory element which act as the binding site for special transcription factors. Chimeric promoters are custom-designed and are induced by various factors or re-primed. Examples of such promoters can be found in WO2000/29592 and WO2007/147395. An example of a suitable terminator is the nos-terminator (Depicker et al., 1982).

Moreover, provided is a cell or host cell comprising the nucleic acid molecule, the polypeptide, or the vector or expression cassette of the present invention. Preferably, the cell comprises the nucleic acid molecule of the present invention as a transgene or as an endogene wherein the nucleic acid molecule being present as endogene is preferably a resistance protein. In this regard the invention also covers cells the genome of which has been modified to encode one or more substitutions according to the inventions in an encogene wherein the endogene encodes a resistance protein. The modifications may be achieved by the use of genome editing technologies as described elsewhere herein. Also encompassed is a plant comprising one or more of such cells or being regenerated from such cell.

The vector or expression cassette may, for example, be introduced into the (host) cell by conjugation, mobilization, biolistic transformation, agrobacterium-conferred transformation, transfection, transduction, vacuum infiltration or electroporation. Methods for vector introduction, as well as methods for the preparation of the vectors and expression cassettes, are familiar to the person skilled in the art (see, for example, Sambrook et al., Molecular Cloning, Cold Spring Harbor Laboratory, 3rd Ed., 2001).

In one embodiment, the host cell may be a prokaryotic cell, e.g., a bacterial cell. In another embodiment, the host cell may be a eukaryotic cell (e.g., a plant cell or a yeast cell). Particularly preferred bacterial host cells are Agrobacterium tumefaciens, A. rhizogenes, and E. coli.

Further provided is a plant or part thereof comprising the nucleic acid molecule of the present invention or comprising the cell of the present invention, and a plant or part thereof regenerated from said cell.

Also provided is a seed or offspring from the plant of the present invention, wherein the seed or the offspring comprises the nucleic acid molecule of the present invention as a transgene or an endogene.

An additional aspect of the invention is seed stock comprising seeds that contain the nucleic acid or acids according to the invention. The nucleic acid or acids according to the invention may be present transgenically or endogenously. The seed stock and the seeds may be technically treated. The invention thus also comprises technically-treated seed stock and technically-treated seeds. The various embodiments of technically-treated seed stock are explained in detail in the following whereby the term seed stock also includes seeds: Technically-treated seed stock may be present in polished form. The outermost layer of the seed is thereby removed, so that the seed assumes a more rounded form. This is helpful in sowing. An optimally uniform shape leads to a uniform distribution of the seed stock grains. Technically-treated seed stock furthermore encompasses pilled seed stock. The seed stock is thereby placed on a pilling mass that protects the seed stock contained therein and leads to a larger mass, such that the pilled seed stock shows a greater resistance capability with regard to wind drift and is thus less susceptible to being blown away by the wind, and, at the same time, a more precise positioning during sowing is enabled. In a preferred embodiment of the invention, all pilled seed stock grains of a batch or unit designated for sale have essentially the same shape and the same mass. Deviations of 5% in diameter and mass are possible. However, the deviations preferably do not exceed 1%. As one of the main components, the pilling mass may contain for example a mineral compound such as clay and/or peat, for example. Additional possible components are cited in U.S. Pat. No. 4,067,141. Moreover, the pilling mass may contain additional chemical agents that positively influence the cultivation in practice. These may here be substances that are counted among fertilizing agents. Furthermore, these may be fungicides, insecticides, and/or antifeedants. The fungicides may be thiram and/or hymexazol and/or other fungicides. The insecticide may be a substance from the neonicotinoid group. The substance from the neonicotinoid group is preferably imidacloprid (ATC Code: QP53AX17) and/or clothianidin (CAS number 210880-92-5). Furthermore, the insecticide may also be cyfluthrin (CAS number 68359-37-5) or beta-cyfluthrin.

The pilled seed stock is a specific embodiment of dressed seed stock. In this context technically-treated seed stock encompasses also the dressed seed stock. However, the invention is not limited to pilled seed stock, but, rather, may be applied with any form of dressed seed stock. The invention thus also relates to dressed seed stock, which includes pilled seed stock, but is not limited to this. Dry dressing, wet dressing, and suspension dressing are thus also encompassed. The dressing may thereby also contain at least one dye, such that the dressed seed stock may be quickly differentiated from undressed seed stock, and, furthermore, good visibility in the environment is ensured after sowing. The dressing may also contain those agrochemicals which are described in the context of the pilling mass. The invention includes thus such dressed seed stock whereby the dressing contains at least one antifeedant such as an insecticide and/or at least one fungicide. Optionally, so called electonical dressing (dressing by application of electric energy) may be applied. However, electronic dressing is not a dressing in the strict sense of the word.

An additional form of technically-treated seed stock is encrusted seed stock. What is known as coating is also spoken of in this context as well as of seed stock treated with a coating. The difference to pilled seed stock is that the seed grains retain their original shape, wherein this method is especially economical. The method is described in EP 0 334 258 A1, for example. An additional form of technically-treated seed stock is sprouted or primed seed stock. Sprouted seed stock is pretreated via a pre-germination, whereas primed seed stock has been pretreated via a priming (“germination”). Pre-germinated and primed seed stock have the advantage of a shorter emergence time. The point in time of the emergence after sowing is, at the same time, more strongly synchronized. This enables better agrotechnical processing during cultivation and especially during the harvest, and, additionally, increases the yield quantity. In pre-germination, the seed stock is germinated until the radicle exits the seed stock shell, and the process is subsequently stopped. In the priming, the process is stopped before the radicle exits the seed stock shell. Compared to pre-germinated seed stock, seed stock that has been subjected to a priming is insensitive to the stress of a re-drying and, after such a re-drying, has a longer storage life in comparison to pre-germinated seed stock, for which a re-drying is generally not advised. In this context, technically pre-treated seed stock also includes primed and re-dried seed stock. The process of pre-germination is explained in U.S. Pat. No. 4,905,411 A. Various embodiments of priming are explained in EP 0 686 340 A1. In addition to this, it is also possible to simultaneously pill and prime seed stock in one process. This method is described in EP 2 002 702 B1. Primed seed stock which is moreover pilled, is encompassed by the present invention.

The technically-treated seed stock may additionally be furnished with one or more of the herbicide resistances explained above. This allows a further-improved agrotechnical cultivation, since the technically-treated seed stock may be deployed on a field that has previously been treated with weed killer, and that therefore is weed-free.

In addition to this, the invention also encompasses a mixture containing the seed stock according to the invention or the seeds according to the invention, and a dressing mass as defined above. The dressing mass is thereby preferably embodied as a pilling mass, as defined above.

With storage of seed stock according to the invention, storage conditions are preferably to be chosen that do not negatively affect the stability or storage life of the seed stock. Fluctuations in humidity may, especially, have a disadvantageous effect here. Part of the invention is a method for the storage of the seed stock in a container that is via simultaneously water-repellent and breathable. Such a container may be designed as a carton. Such a carton may optionally possess an inner vapor barrier. If the carton is designed as a duplex carton, its stability increases. A seed stock according to the invention that includes such a container and such a carton, or technically-treated seed stock according to the invention, is likewise part of the invention. It is likewise part of the invention to contain seed stock according to the invention or technically-treated seed stock according to the invention in such a carton.

In one embodiment, the plant according to the invention is a hybrid plant or a double haploid plant. Hybrid plants and double haploid plants do not occur in nature and cannot be isolated from nature. In a further embodiment of the plant according to the invention, the nucleic acid molecule according to the invention is present in heterozygous or homozygous form. In the case of a hybrid plant, the nucleic acid molecule may also be present in hemizygous form. The invention also encompasses hybrid seeds and double haploid seeds which contain a nucleic acid according to the invention or a polypeptide according to the invention.

The term “transgene” (or “transgenic”) is understood to mean that the respective gene is an exogenous gene that was introduced into the plant. The exogenous gene may be derived from a species other than the plant species into which it is introduced. Alternatively, the respective gene may be a gene already present in the plant species into which it is introduced, so that one or more additional copies of said gene are present as a result introducing the transgene.

The present invention also provides a method for increasing a plant's resistance towards at least one plant pathogen. In one embodiment, said method comprises the steps of introducing the nucleic acid molecule of the present invention into the genome of at least one cell of a plant and regenerating a plant from that cell. This may be done via homology-directed repair or homologous recombination for example by the use of gene editing technology as stated elsewhere herein. Alternatively, said method comprises the steps of transforming a plant cell with a nucleic acid molecule of the present invention, or the vector or the expression cassette of the present invention, and regenerating a plant from that cell. The methods for increasing a plant's resistance towards at least one plant pathogen may further comprise the step of causing the expression of the nucleic acid molecule in the plant.

“Introducing” in the meaning of the present invention includes stable integration by means of transformation including Agrobacterium-mediated transformation, transfection, microinjection, biolistic bombardment, insertion using gene editing technology like CRISPR systems, TALENs, zinc finger nucleases or meganucleases, homologous recombination, modification of endogenous gene using random or targeted mutagenesis like TILLING or gene editing, introgression using breeder's methods, etc.

Moreover, the invention comprises a method for the production of a plant comprising a gene encoding a resistance protein wherein the resistance protein has a decreased or reduced induction of cell death or reduced cell death-inducing activity during a hypersensitive response comprising the following steps:

a) Providing a plant or cell thereof comprising an endogenes gene encoding a resistance protein

b) Modifying the coding sequence of the endogenes gene to encode one or two or three or more amino acid substitutions within the amino acid sequence of the resistance protein thereby encoding a resistance protein having a decreased or reduced induction of cell death or reduced cell death-inducing activity during a hypersensitive response

c) Optionally regenerating a plant of the cell

The modification may be achieved by the use of CRISPR systems, TALENs, zinc finger nucleases or meganucleases, homologous recombination, modification of endogenous gene using random or targeted mutagenesis like TILLING. The resistance gene may be one of the resistance genes given elsewhere herein and/or a resistance gene structurally defined by the disclosure of the sequence listing. The encoded amino acid substitution. The modification may encode an amino acid substitution as stated elsewhere herein. Especially the amino acid substitution may encode one of the amino acid substitutions as disclosed herein for the resistance gene R3a.

Furthermore, if the resistance genes belong to the class of CC_{DAE}-NBS-LRR resistance genes the amino acid substitution may be a substitution as defined herein in the context of the method for the modification of an R-gene belonging to the CC_{DAE}-NBS-LRR resistance gene class. The invention is also related to a plant obtainable by this method.

The regeneration of organisms out of cells is explained in various standard references of the cell related biology. The regeneration of plants is for example disclosed in the standard reference “Plant biotechnology: comprehensive biotechnology, second supplement” (Michael W. Fowler, Graham Warren, Murray Moo-Young—Pergamon Press—1992). An example concerning especially the regeneration of Beta vulgaris is given in Lindsey, K., and P. Gallois. “Transformation of sugarbeet (Beta vulgaris) by Agrobacterium tumefaciens.” Journal of experimental botany 41.5 (1990): 529-536.

As the invention is related to biological processes the exact effect may be dependend on the avirulence and/or resistance protein chosen, the substitutions included in the protein(s) and the plant species in which the invention is put into practice. For achieving the best possible results, it may be worth to test several avirulence and/or resistance proteins in a specific plant species and to test different substitutions or substitution combinations within the avirulence and/or resistance protein. Therefore, the invention also comprises a method for testing of resistance and/or avirulence having different substitutions in a plant comprising the following steps:

1) Providing a plant

2) Transforming the plant stably or transiently with

a) a nucleic acid sequences encoding at least two proteins wherein the proteins are resistance and/or avirulence proteins

b) at least two nucleic acid sequences each encoding a protein wherein the protein is a resistance and/or avirulence protein

wherein each protein comprises different substitutions according to the invention

3) Assessing the resistance resulting from the transformation of one of the proteins under step 2)

4) Optionally selecting the plant comprising the protein which comprises the substitution(s) which showed the best resistance or optionally selecting the protein which comprises the substitution(s) which showed the best resistance.

The assessment of the resistance can be done by infecting the plant with a pathogen and quantifying the amount of cell death and detecting the presence of a hypersensitive reaction. The pathogen to be used in the test is preferably a fungus and particularly preferably the pathogen from which the avirulence protein (if an avirulence protein has been tested) originates. The invention also relates to the protein which is selected or selectable by the testing method given above.

Further, the present invention provides a method for the identification of a plant comprising the nucleic acid molecule of the present invention, said method comprising the steps of isolating DNA from the plant of the present invention or part thereof, and performing a polymerase chain reaction using the isolated DNA as a template, thereby amplifying the nucleic acid molecule of the present invention or a part thereof. The method may further comprise the step of detecting the presence of the nucleic acid molecule or a part thereof and thereby identifying the plant. For this approach it is suitable to perform the polymerase chain reaction in the presence of at least one molecular marker which allows due to its chemical structure the differentiation of single nucleotide polymorphisms (SNPs).

The present invention further provides an oligonucleotide which may be used, for example, as a primer in the identification method of the present invention, wherein the oligonucleotide is able to hybridize to a nucleic acid molecule of the present invention. Preferably, the nucleotide sequence of the oligonucleotide is identical to a part of the nucleic acid molecule of the present invention and has a length of at least 15 nucleotides.

Furthermore, the oligonucleotide or molecular marker according to the invention may be connected to a fluorescent dye in order to generate a fluorescence signal, e.g., under excitation via light of the corresponding wavelength. The fluorescent dye may be fluorochrome. The oligonucleotides according to the invention may be coupled with other compounds that are suitable for generating a signal. Such oligonucleotides or molecular markers do not occur in nature and cannot be isolated from nature. The following is executed to produce such marked oligonucleotides: DNA may be marked bio-orthogonally. For this, DNA may be marked in vivo or in vitro with nucleoside analogs, which, for example, may subsequently be coupled with a fluorophore per Staudinger reaction. In addition to this, DNA may also be chemically provided with fluorophores. Oligonucleotides may be marked via a phosphoramidite synthesis with fluorophores that, for example, are used in QPCR, DNA sequencing, and in situ hybridization. Furthermore, DNA may be generated enzymatically in the course of a polymerase chain reaction with fluorescent nucleotides, or be marked with a ligase or a terminal deoxynucleotidyl transferase. DNA may also be detected indirectly via a biotinylation and fluorescent avidin. For couplings, fluorescein, fluorescent lanthanides, gold nanoparticles, carbon nanotubes, or quantum dots, among other things, are used as fluorophores. One of the most commonly used fluorescent substances is FAM (carboxyfluorescein). Consequently, oligonucleotides and, in particular, primers that possess a FAM marking are encompassed by the invention. FAM is preferably present as 6-FAM, wherein—depending upon the desired wavelength of the emission and excitation—other FAM variants, e.g., 5-FAM, may, however, also be used. Examples of additional fluorescence markers are AlexaFluor, ATTO, Dabcyl, HEX, Rox, TET, Texas Red, and Yakima Yellow. Depending upon the field of use, the oligonucleotides may be furnished with modifications of the bases or of the sugar phosphate spine. Among these are, among others, amino-dT, azide-dT, 2-aminopurine,5-Br-dC, 2′-deoxyinosine (INO), 3′-deoxy-A, C, G, 5-Met-dC, 5-OH-Met-dCN6-Met-dA, and others.

A kit comprising at least one of the oligonucleotide of the present invention is also provided. The kit may also contain a pair of the oligonucleotides of the present invention.

Furthermore, the present invention also relates to a marker chip (“DNA chip” or microarray) which contains at least one oligonucleotide according to the invention that is suitable for detection. The marker chip is suitable for application in one or more detection methods according to the invention.

Moreover, provided is a method for the reduction of plant pathogen infestation or the formation of necrotic lesions during the cultivation of plants, comprising the steps of (i) planting the plant or part thereof of the present invention, or the seed of the present invention in a cultivation area, and (ii) growing the plant or a germling resulting from the seed.

The nucleic acid according of the present invention may be used for conferring or increasing resistance towards at least one plant pathogen to a plant or a part thereof.

Furthermore, the present invention may be used to engineer any CC-NBS-LRR resistance gene and/or its corresponding avirulence gene using the promoter induced co-expression strategy for the development of pathogen-resistant plants. The technical application of the invention is not restricted to fungal disease but may also be used to develop plants resistant to insects, bacteria, nematodes and/or viruses if an appropriate pathogen-inducible promoter is selected. Suitable resistance proteins and corresponding avirulence proteins are listed in Tab. 1.

The present invention also comprises the use of a nucleic acid according to the invention for conferring or increasing resistance towards at least one plant pathogen to a plant or a part thereof.

The present invention also comprises an oligonucleotide for use as a primer in a method for the identification of a plant according to the present invention as described above, wherein the oligonucleotide is able to hybridize to a nucleic acid molecule according to the present invention. The nucleotide sequence of the oligonucleotide may be identical to a part of the nucleic acid molecule according to the invention and may have a length of at least 15 nucleotides.

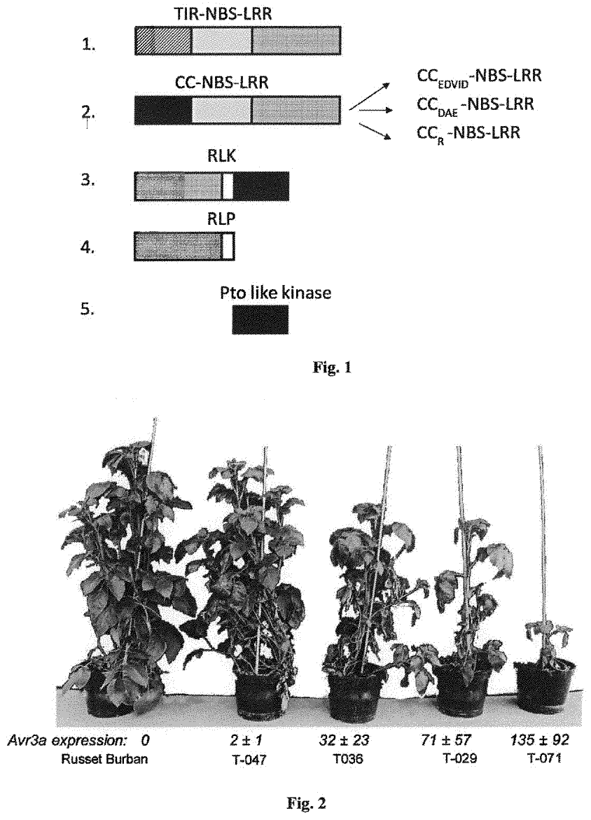

Furthermore, the present invention is also related to a method for the modification of an R-gene belonging to the CC_{DAE}-NBS-LRR resistance gene class (FIG. 1). The method is suitable for the production of a gene encoding a modified resistance protein wherein the modified resistance protein leads to a reduced cell death in comparison to the resistance protein lacking the modification. The method comprises the following steps:

a) Providing a gene encoding an R-gene belonging to the CC_{DAE}-NBS-LRR resistance gene class

b) Modifying the nucleic acid sequence to generate an amino acid substitution selected from the group consisting of:

I) Substitution of a basic, positively charged amino acid by an acidic negatively charged amino acid or an aliphatic amino acid in the CC-domain of the protein

II) Substitution of D or E in the DAE motif by a basic, positively charged amino acid like K or R or by an aliphatic amino acid like V, I or L

III) Substitution of one of the two conserved acidic, negatively charged amino acids at the C-terminus of the CC-domain by a polar amino acid like N or Q.

Examples for acidic, negatively charged amino acids are for example aspartic acid (D) or glutamate (E). Examples for basic, positively charged amino acids are for example histidine, lysine or arginine.

In a preferred embodiment of this method the substitution is a substitution according to I) wherein the substitution is selected from: K30E, R31G and/or K119D within SEQ ID NO 263; K35E and/or K37M within SEQ ID NO 269 or the substitution is a substitution according to II) wherein the substitution is selected from: E61V and/or E61K within SEQ ID NO 269 or the substitution is a substitution according to III) wherein the substitution is selected from E135N and/or E136N within SEQ ID NO 269. The present invention is also related to a protein obtainable by this method. The substitution according to step b) may be done in vitro. Furthermore, it is possible to perform this step by gene editing approaches like TALENS, Zinc Finger Nucleases and CRISPR nuclease including Cas9, CasX, CasY or Cpf1 nuclease.

In a further aspect of this invention the following method is provided: A method for increasing a plant's resistance towards at least one plant pathogen comprising:
(i) integrating the nucleic acid molecule according to any one of claims 1 and 3-8 into the genome of at least one plant cell and regenerating a plant from that cell; or
(ii) transforming a plant cell with a nucleic acid molecule according to any one of claims 1 and 3-8, or the vector or the expression cassette according to claim 2, and regenerating a plant from that cell

wherein the nucleic acid molecule according to step (i) or (ii) encodes a resistance protein comprising the amino acid sequence according to SEQ ID NO 269 and/or an avirulence protein comprising the amino acid sequence according to SEQ ID NO 2 or according to SEQ ID NO 267. Optionally the plant cell according to step (i) or (ii) is the cell of a plant belonging to the family of Poaceae or Fabaceae.

### EXAMPLES

The following examples are provided for illustrative purposes only and are not construed as limiting the present invention.

### Example 1: Molecular Stack of R3a Resistance Gene and Avr3a^{KI} Avirulence Gene

A molecular stack of the native R3a resistance gene of potato and the Avr3a^{KI} gene was constructed (FIG. 19) and transformed into the potato cultivar “Russet Burbank”. The expression of the R3a gene was under control of the endogenous R3a promoter, and the Avr3a expression was controlled by the pathogen-inducible synthetic promoter 2xS-4xD-NpCABE which is comprised in SEQ ID NO 270. Transgenic R3a-Avr3a^{KI} lines were generated and transferred into the greenhouse as rooted in-vitro plants. First, the plantlets developed well in soil, showing no difference to the non-transgenic Russet Burbank. However, 3-4 weeks after transfer to the greenhouse, spontaneous necrosis of leaf tissue, abortion of leaves and stunted growth of the plants were observed.

TABLE 3
Infected potato leaves of T-047, T-036, T-029 and T-071 show
reduced sporangia production of P. infestans in comparison to
Russet Burbank but severe disturbance of plant development

| | Sporangia (%) | | | Sporangia (%) | Plant |
|---|---|---|---|---|---|
| | Test: | | | Mean of | phenotype |
| | 1 | 2 | 3 | 3 tests | |
| Russet | 100 | 100 | 100 | 100 ± 0 | normal |
| Burbank | | | | | |
| (control) | | | | | |
| T-047 | 61 | n.d. | n.d. | 61 | 30% size reduction |
| | | | | | Spontaneous leaf |
| | | | | | necrosis |
| T-036 | n.d. | 16* | 59 | 37 | 50% size reduction |
| | | | | | single leaf fall |
| T-029 | 21* | 11* | 43* | 25 | 50% size reduction |
| | | | | | single leaf fall |
| T-071 | n.d. | n.d. | n.d. | n.d. | 80% size reduction |
| | | | | | severe leaf fall |

The sporangia were washed off from the infected potato leaves (n=8) at 6 dpi and the sporangia numbers were determined by a particle counter. The number of sporangia of Russet Burbank was set to 100. In total 3 independent resistance assays were performed. Differences between samples were assessed by using analysis of variance (ANOVA). (*=Significant different according ANOVA to Russet Burbank p<0.05. n.d.=not determined.)

Four R3a-Avr3a^{KI} lines, which were analyzed in detail, showed a reproducible gradient of disintegration (FIG. 2). AqRT-PCR analysis revealed that the level of the negative phenotype of adult plants correlated with the expression of the Avr3a^{KI} gene. Expression of the Avr3a^{KI} gene was also detectable in healthy young plants, and the Avr3a expression was even higher than in adult plants (FIG. 2). This indicated that the resistance gene R3a is developmentally regulated and the resistance mechanism is not active in young plants.

Therefore, on the basis of these results it can be concluded that the direct transfer of the naturally occurring late blight resistance gene R3a from potato (lacking a substitution) and the naturally occurring Avr3aKI gene of Phytophthora infestans (lacking a substitution) under the control of a synthetic pathogen-inducible promoter leads to severe growth retardation and leaf damage of the plant even in the absence of the pathogen. During greenhouse cultivation, the pathogen-inducible promoter is activated by unknown developmental factors and the highly active Avr3aKI protein induces cell death. The results show that the approach of direct transfer is agronomically impractical.

### Example 2: Modification of R3a Gene Activity

In order to find a balanced level of gene expression, which does not harm the plant in the absence of the pathogen, mutagenized versions of the R3a gene were developed and successfully tested in a transient assay.

A comparison of the CC-domain of R3a with the CC_{DAE}-domains of the sugar beet R-genes BvKWS3_165 and Bv123 revealed that the DAE and EDVID motifs are conserved (FIG. 3). The R3a gene is therefore a member of the CC_{DAE}-NBS-LRR resistance gene class (FIG. 1). A loss-of function analysis of the CC_{DAE}-domain of the R-gene BvKWS3_165 according to SEQ ID NO 263 showed that the K30E, R31G, E96V, K119D, L153P modification, a D and E exchange in the DAE motif and the E143N and E144N modification of two conserved acidic amino acids at the C-terminus impaired the cell death inducing activity. Based on this knowledge, allelic R3a variants with reduced cell death inducing activity were generated in the CC_{DAE}-domain of the CC-NBS-LRR resistance gene.

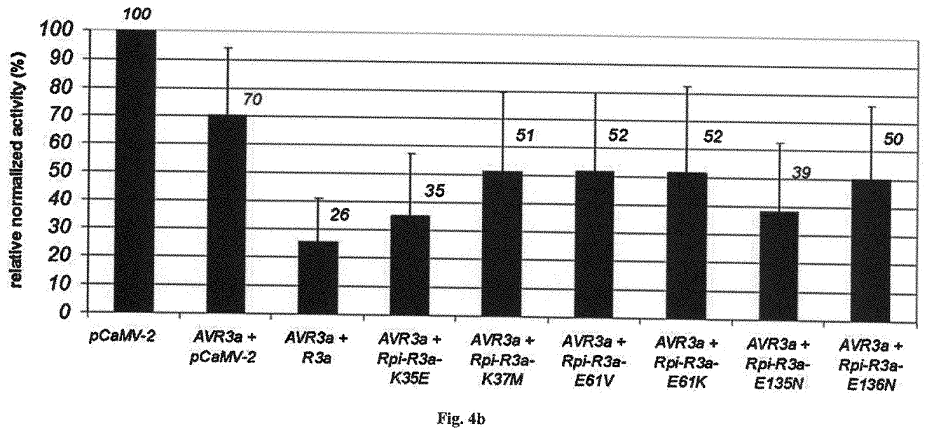

As a first rule, the replacement of a basic, positively charged amino acid with an acidic, negatively charged amino acid or an aliphatic amino acid in the CC-domain impairs the HR-inducing activity, as shown for K30E, R31G and K119D of BvKWS_165 (compare to SEQ ID NO 263) (FIG. 4a). This rule was applied to reduce R3a activity by a K35E and K37M exchange in the R3a protein (compare to SEQ ID NO 265) (FIG. 4b).

As a second rule, the replacement of D or E in the DAE motif with a basic, positively charged amino acid (K, R) or an aliphatic amino acid (V, I, L) reduces R-gene activity, as shown by an E61V and E61K exchange for R3a (compare to SEQ ID NO 265) (FIG. 4b).

As a third rule, the replacement of one of the two conserved acidic, negatively charged amino acids at the C-terminus of the CC-domain with a polar amino acid (N, Q) reduces cell death, as shown for the E135N and E136N of R3a (compare to SEQ ID NO 265) (FIG. 4b).

These mutagenized versions can be used to generate co-expression lines without a detrimental phenotype.

### Example 3: Modification of Avr3a Activity

The Avr3a gene of P. infestans is essential for virulence (Bos et al., 2009). Among other existing alleles, two variants seem to have an outstanding function. They are present either as an Avr3a_K59I82 (Avr3a^{KI}) according to SEQ ID NO 1 or as an Avr3a_E59M82 (Avr3a^{EM}) allele according to SEQ ID NO 12 in all strains examined. The Avr3a^{KI} allele activates R3a resistance gene dependent innate immunity, whereas Avr3a^{E}M was not recognized by the R3a gene (Amstrong et al. 2005).

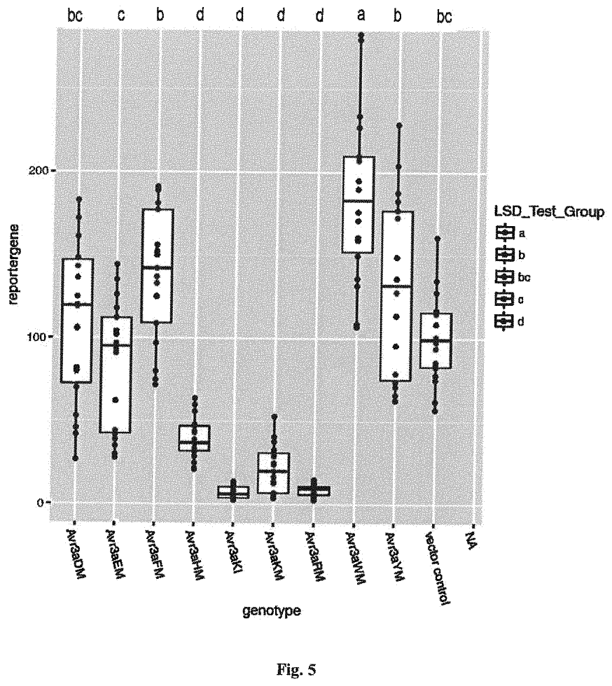

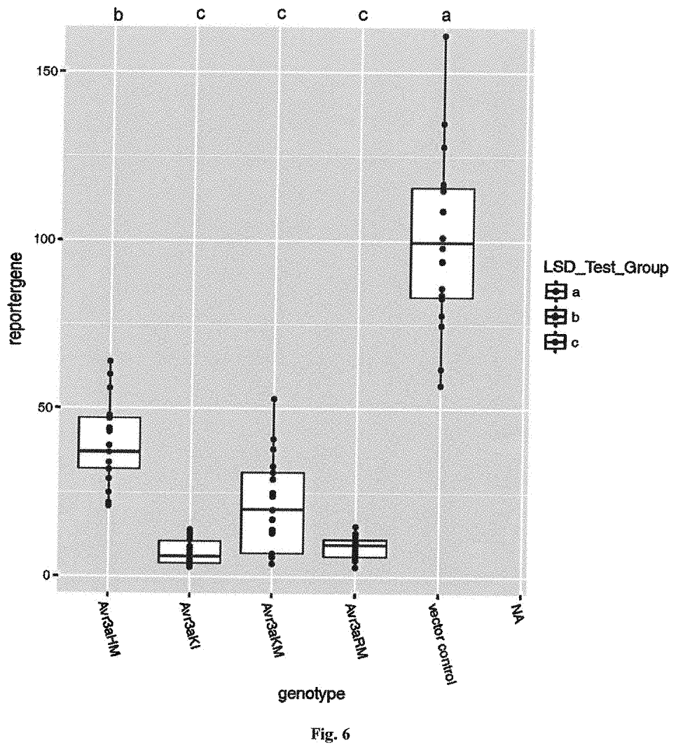

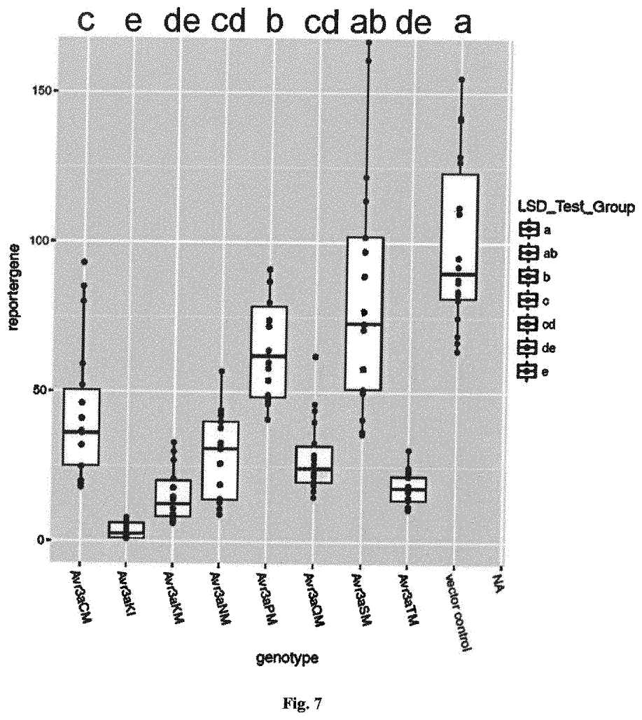

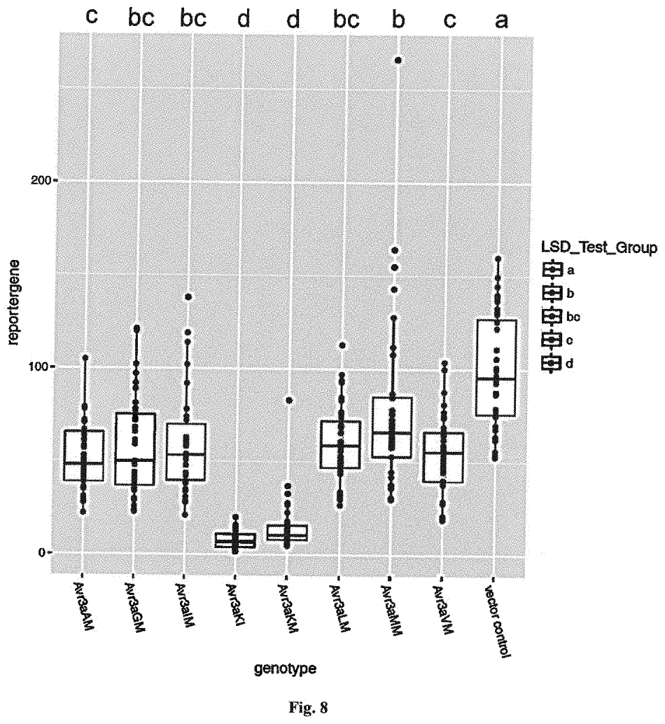

We tested the 20 Avr3a^{XM} (x=each of the 20 natural amino acids) alleles as given in Tab. 2 by ballistic transient assays in leaves of potato expressing the R3a gene. Fourteen alleles—Avr3a^{HM} Avr3a^{KM}, Avr3a^{RM}, Avr3a^{CM}, Avr3a^{NM}, Avr3a^{PM}, Avr3a^{QM}, Avr3a^{TM}, Avr3a^{AM}, Avr3a^{GM} Avr3a^{M}, Avr3a^{EM}, Avr3a^{MM}, Avr3a^{VM}—showed statistically significant cell death induction compared to the vector control (FIGS. 5-8). Twelve of these alleles—Avr3a^{HM}, Avr3a^{CM} Avr3a^{NM}, Avr3a^{PM} Avr3a^{QM}, Avr3a^{T}M, Avr3a^{AM}, Avr3a^{GM}, Avr3a^{IM}, Avr3a^{EM}, Avr3a^{MM}, Avr3a^{VM}—induced statistically significant less cell death compared to the avirulent Avr3a^{KI} allele (FIGS. 5-8). This result showed that Avr3a alleles were identified which trigger less cell death in potato than the avirulent allele Avr3a^{KI}, and that these alleles differ in the level of cell death induction.

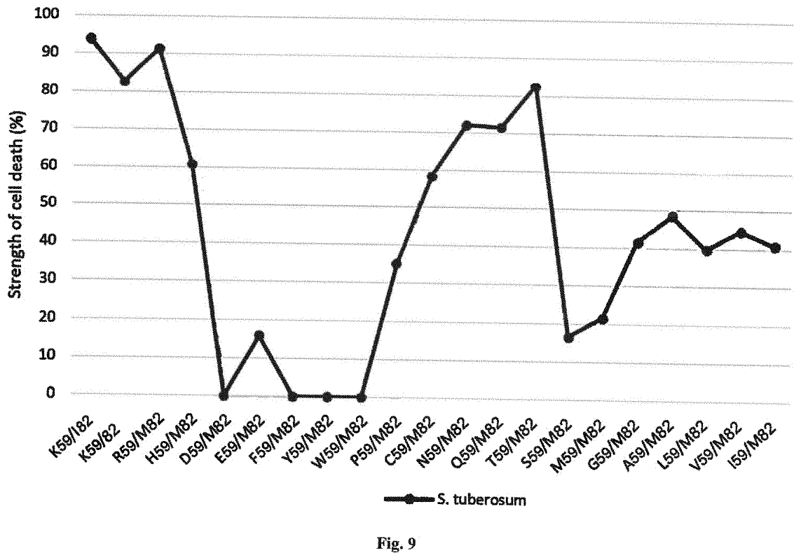

The sequences given in Table 2 were tested in potato leaves (FIG. 9). The alleles Avr3a, Avr3a^{KM} Avr3a^{RM} triggered a strong cell death in potato, whereas the alleles Avr3a^{DM}, Avr3a^{FM} Avr3a^{YM} and Avr3a^{WM} were not recognized by R3a. However, other alleles (like Avr3a^{HM} Avr3a^{CM}, Avr3a^{NM}, Avr3a^{PM} Avr3a^{SM}, Avr3a^{T}M, Avr3a^{AM}, Avr3a, Avr3a^{LM}, Avr3a^{MM} and Avr3a^{VM}) induced surprisingly low cell death in potato, and the alleles Avr3a^{PM} and Avr3a^{GM} revealed a surprisingly strong cell death induction.

### Example 4: Enhanced Late Blight Resistance

We selected three gain-of-function mutants with moderate level of cell-death inducing activity. Compared to the avirulent allele Avr3a^{KI} (94% cell death), the cell death inducing activity of Avr3a^{IM}, Avr3a^{LM} and Avr3a^{GM} was 41%, 40% and 42%, respectively. The three AVR3a alleles were each combined with the synthetic 2xS-4xD-NpCABE promoter. The promoter-Avr3a cassettes were transformed into the potato cultivar Hermes, which contained the corresponding R3a resistance gene.

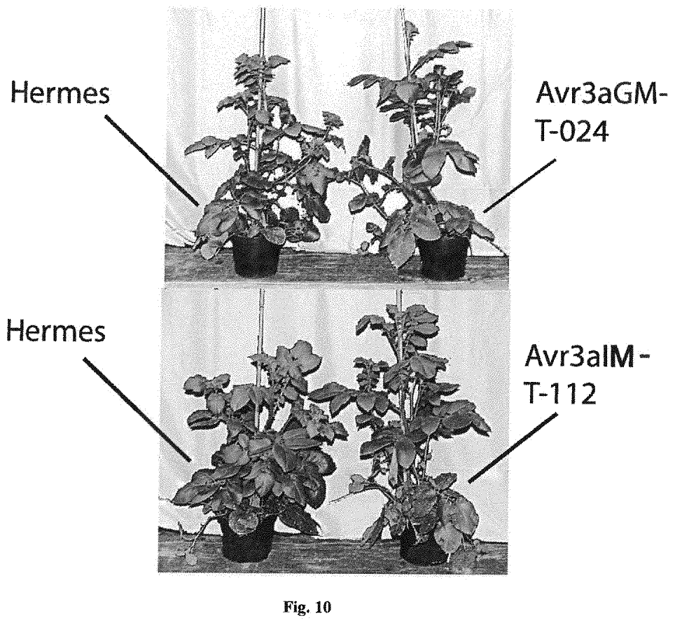

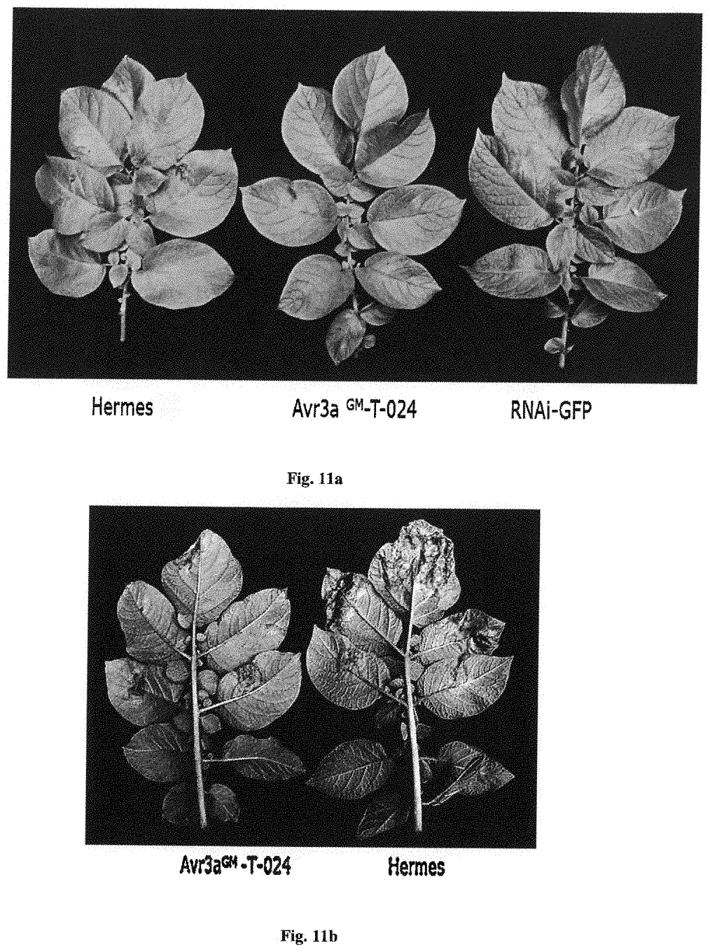

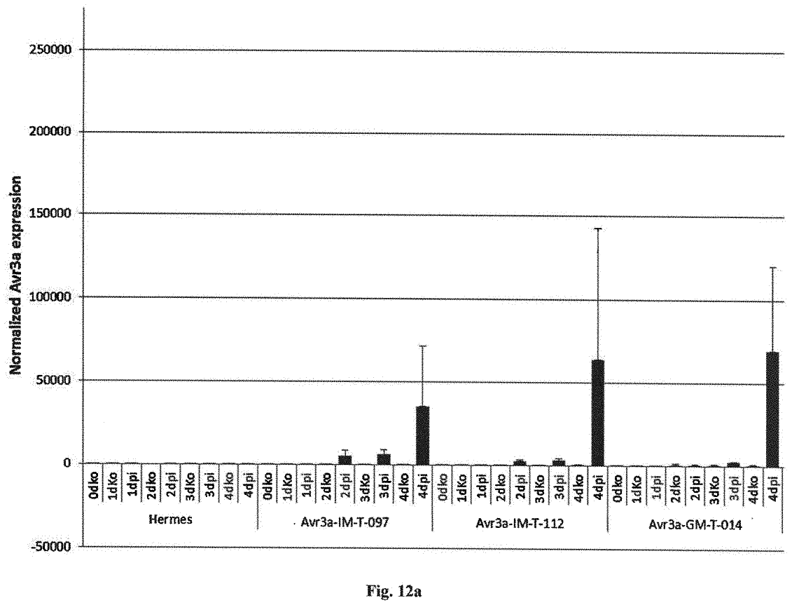

Eight independent transgenic potato lines of each modified Avr3a gene were transferred twice to the greenhouse. The lines were analyzed for a change of the phenotype and fungal resistance. With the exception of two lines, all lines showed the same vegetative growth phenotype as Hermes. One Avr3a^{IM} line showed some growth retardation and the leaves of one Avr3a^{LM} line revealed some micro-necrotic lesions. However, also these plants were viable. A negative phenotype was not detectable for lines were Avr3a^{GM} was present (FIG. 10).

Both sets of plants were tested several times for late blight resistance in a detached leaf assay (DLA). The fungal resistance was determined by a visual scoring of symptoms (5 dpi) and the measurement of sporangia produced in the lesions (6 dpi). Especially the sporangia production is important with respect to the epidemiology of P. infestans. The highest level of resistance improvement was observed for the line Avr3a^{GM}-T-024. This line showed a visible reduction of disease symptoms (FIG. 11a+11b) and only 40% sporangia production compared to the non-transgenic cultivar Hermes and a transgenic control in 5 independent resistance assays with two different sets of plants (Table 4). The line Avr3a^{GM}-T-014 and the line Avr3a^{IM}-T-112 also showed enhanced late blight resistance, as determined by 66% and 71% sporangia production (Table 4). No reproducible resistance improvement without a negative phenotype was detected for the Avr3a^{LM} lines. This result indicates that a reduction of cell-death inducing activity of the Avr gene is a promising strategy to circumvent the detrimental effects of uncontrolled R-Avr gene expression. The maximal possible level of resistance improvement is not known since the analysis was confined to 8 randomly selected lines.

TABLE 4
Infected potato leaves of Avr3a^{IM}-T-112, Avr3a^{GM}-T-014 and
Avr3a^{GM}-T-024 show reduced sporangia production of P. infestans
in comparison to Hermes and a transgenic control (RNAi-GFP).

| | Plant set 1 | | | Plant set 1 | | | Plant set 2 |
|---|---|---|---|---|---|---|---|
| | Sporangia (%) | | | Sporangia (%) | Plant set 2 | | Sporangia (%) |
| | Test: | | | Mean of | Sporangia (%) | | Mean of |
| | 1 | 2 | 3 | 3 tests | Test 1 | Test 2 | 2 tests |
| Hermes (control) | 100 | 100 | 100 | 100 ± 0 | 100 | 100 | 100 ± 0 |
| RNAi-GFP (transgenic control) | n.d. | n.d. | n.d. | n.d. | 102 | 112 | 107 ± 5 |
| Avr3a^{IM}-T-097 | 22 | 89 | 71 | 61 ± 28 | 105 | 86 | 95 ± 10 |
| Avr3a^{IM}-T-112 | 30 | 59 | 96 | 62 ± 27 | 91 | 62 | 76 ± 15 |
| Avr3a^{GM}-T-014 | 26 | 65 | 77 | 56 ± 22 | 87 | 26* | 76 ± 31 |
| Avr3a^{GM}-T-024 | 25 | 31* | 63* | 40 ± 17 | 54 | 28* | 41 ± 13 |
| Avr3a^{LM}-T-013 | 44 | 39 | 81 | 52 ± 19 | 88 | 130 | 109 ± 21 |
| Avr3a^{LM}-T-082 | 96 | 153 | | 126 ± 29 | 159 | 82 | 120 ± 39 |

The sporangia were washed off from the infected potato leaves (n=8) at 6 dpi and the sporangia numbers were determined by a particle counter. The number of sporangia of Hermes was set to 100. In total 5 independent resistance assays were performed with 2 different sets of plants. The black highlighted line Avr3a^{GM}-T-024 showed the highest level of sporangia reduction in all 5 assays. Differences between samples were assessed by using analysis of variance (ANOVA). (*=Significant different according ANOVA to Hermes p<0.05. n.d.=not determined.)

### Example 5: Avr3a Gene Expression Correlates with Resistance

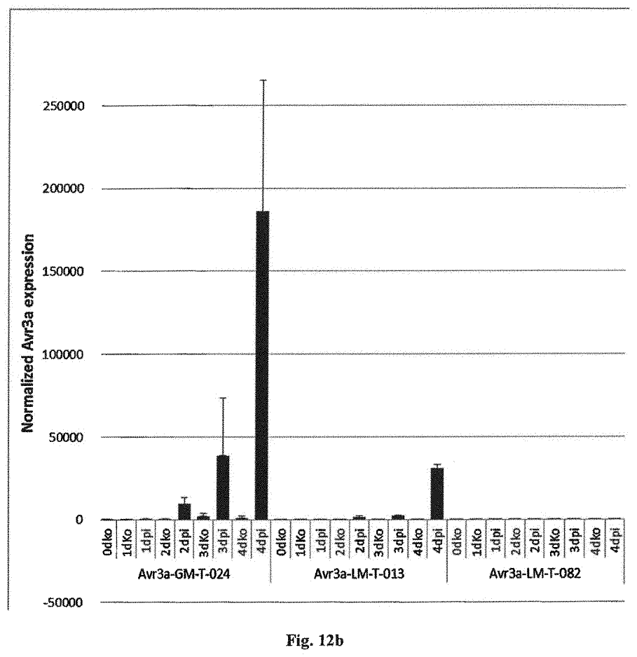

The expression of the recombinant Avr3a gene of 6 transgenic lines and Hermes was measured by qRT-PCR analysis using the primers S53169 according to SEQ ID NO 278 and S3170 according to SEQ ID NO 279. Avr3a expression was normalized against the expression of the potato gene StMCB7 using the primers S1494 according to SEQ ID NO 280 and S1495 according to SEQ ID NO 281. Leaves were infected by P. infestans as described for the DLA, and RNA was extracted from inoculated and control leaves 0, 1, 2, 3 and 4 days after inoculation (dpi). The expression analysis showed a correlation between Avr3a gene expression and resistance improvement. The strongest Avr3a expression could be detected for the resistant line Avr3a^{GM}T024. The absolute expression of the Avr3a^{GM} gene in the resistant line was superior to the other lines at 2, 3 and 4 dpi (FIGS. 12a and 12b). Avr3 agene expression was nearly undetectable for line Avr3a^{LM}-T-082, which did not show resistance improvement in any assay.

### Example 6: Modification of Avr3a Gene Increases Threshold for Cell Death Induction

The transgenic lines T-047, T-036, T-029 and T-071, which were transformed with the R3a-Avr3a^{KI} genes, showed leaf abortion and different stunted growth in the greenhouse dependent on the Avr3a^{KI} expression level (FIG. 2). Expression analysis by qRT-PCR showed that even the lowest detected Avr3a^{KI} gene expression of 1.8 relative units in leaves of T-047 triggers spontaneous cell death in the absence of a pathogen.

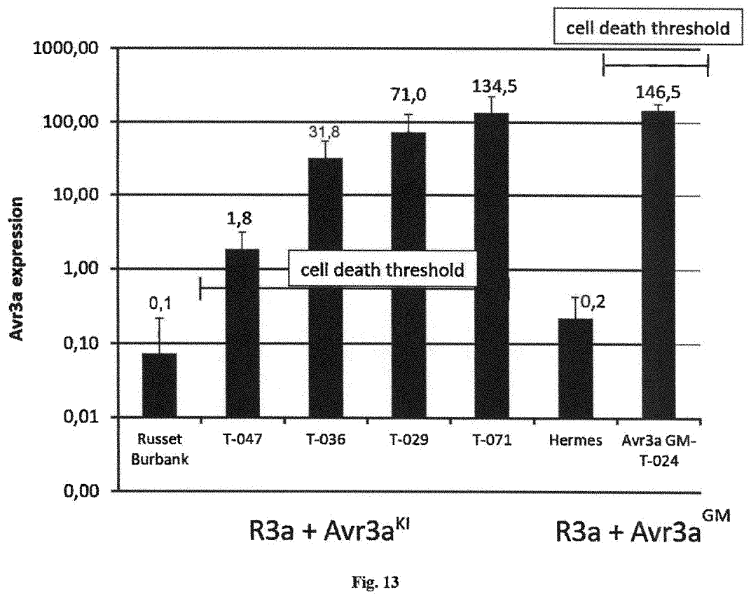

The late blight resistant line Avr3a^{GM}-T-024 showed a background expression level of 146.5 relative units in the absence of a pathogen without any detrimental effect for the vegetative growth (FIGS. 10, 12a, 12b). These results demonstrated that the threshold for cell death induction was at least 100-fold increased by application of the modified Avr3a^{GM} gene (FIG. 13).

### Example 7: Enhanced Early Blight Resistance

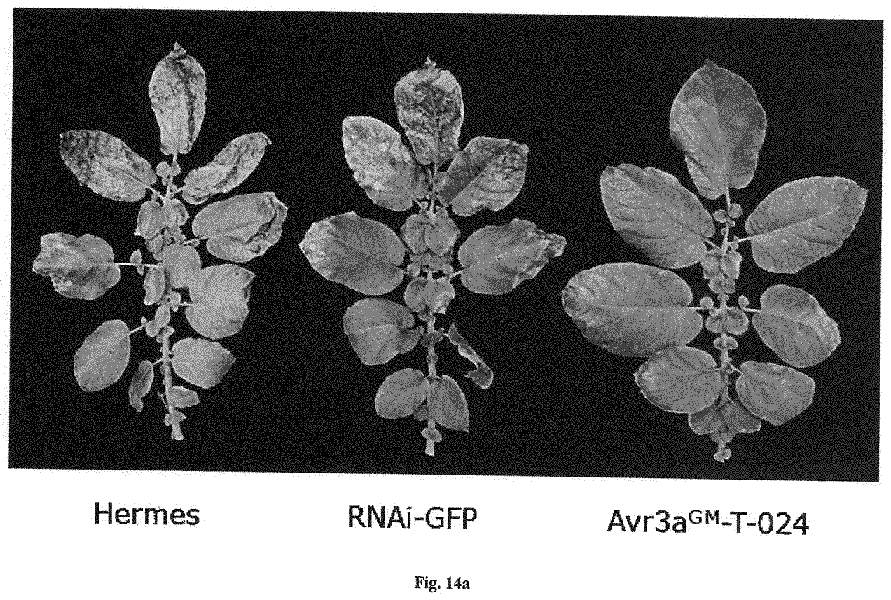

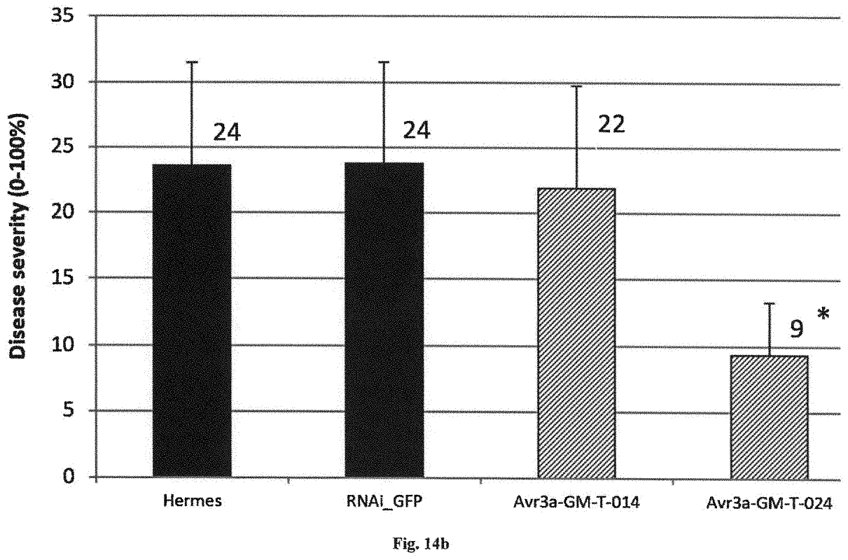

The potato cultivar Hermes is very susceptible to Alternaria solani, the causal agent of early blight, as observed in field trials in the USA. Adult plants of both plant sets also showed early blight symptoms under greenhouse conditions. In both experiments, the late blight resistant line Avr3a^{GM}-T-024 revealed enhanced early blight resistance. Finally, an early blight disease scoring was made for the 2^{nd} plant set. Avr3a^{GM}-T-024 showed only 40% disease symptoms compared to Hermes and the transgenic control, which had been transformed with an RNAi construct directed against the GFP gene (FIG. 14a+14b).

### Example 8: Induction of Innate Immunity in Corn, Soybean and Wheat by R3a and Avr3a

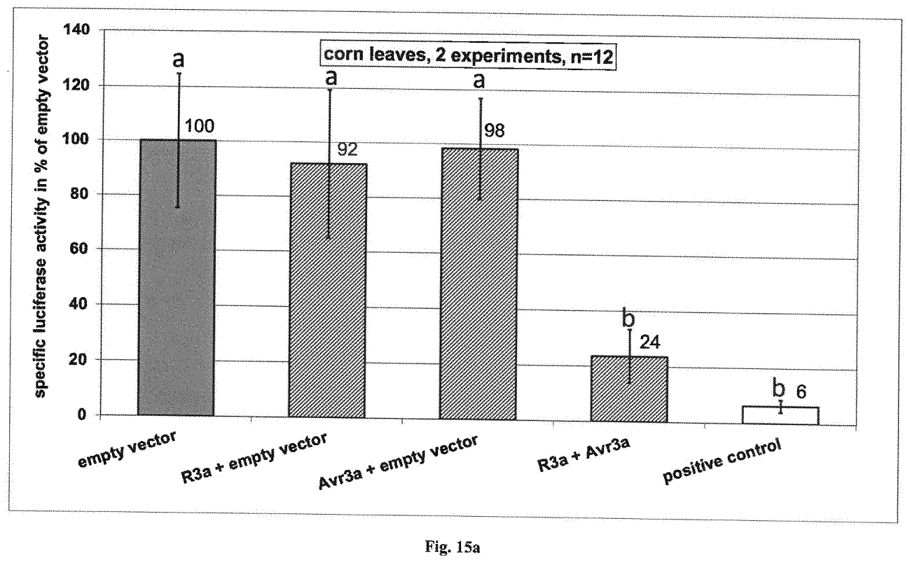

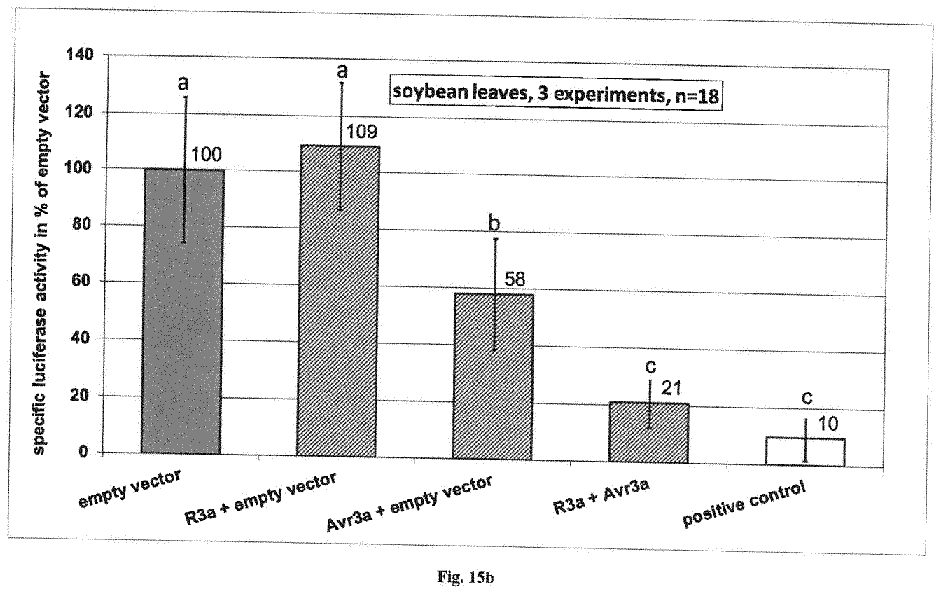

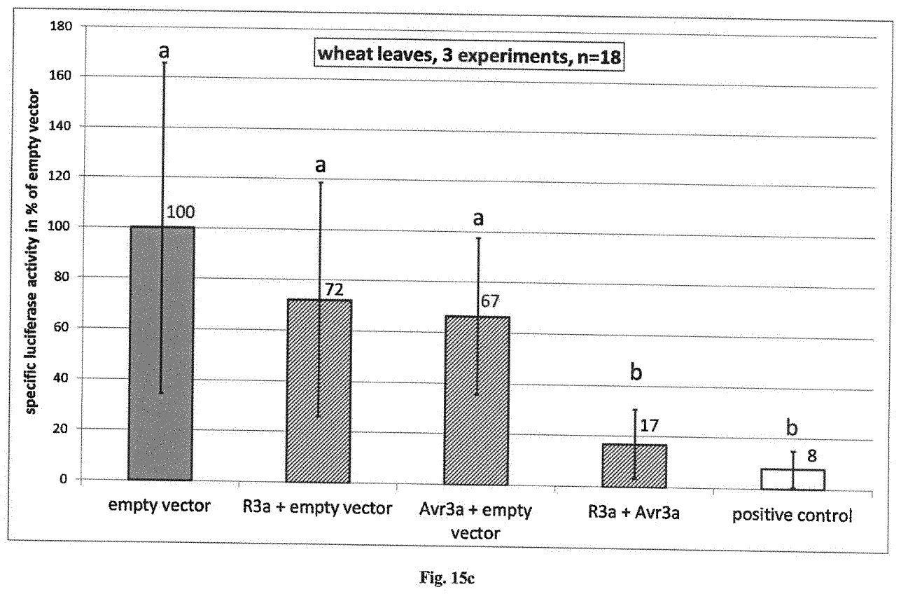

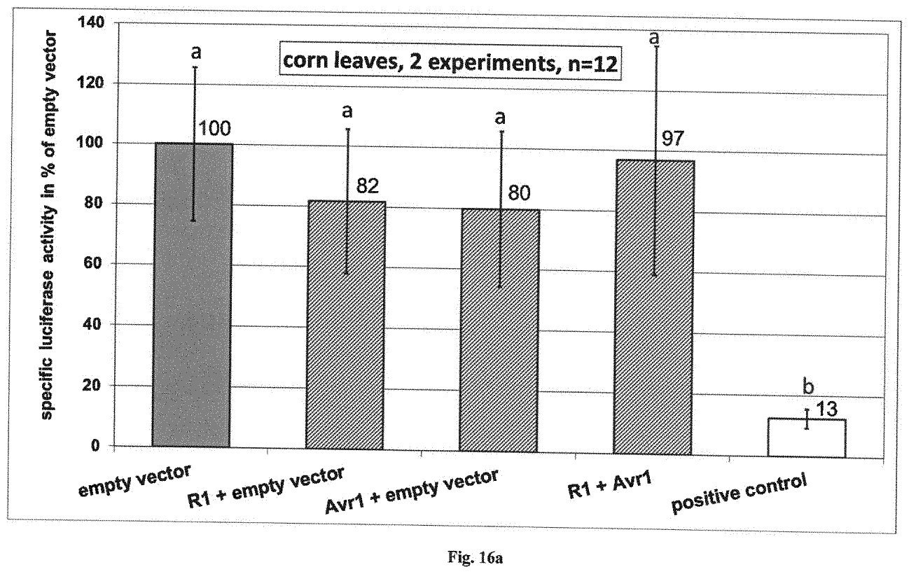

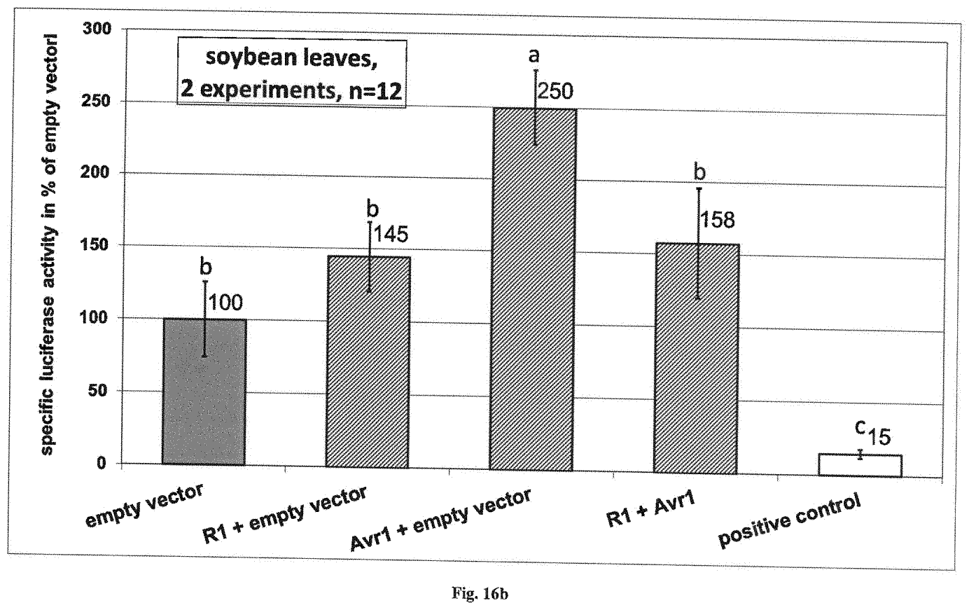

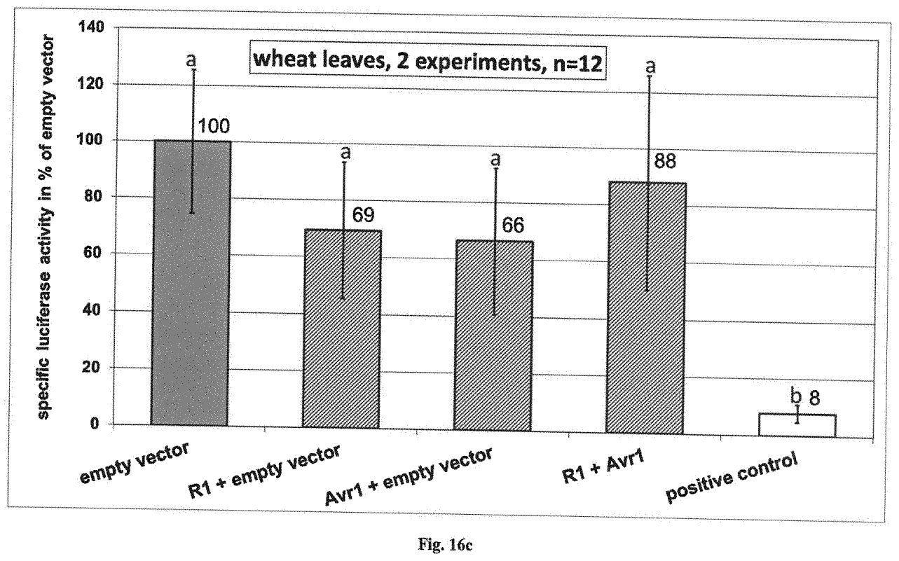

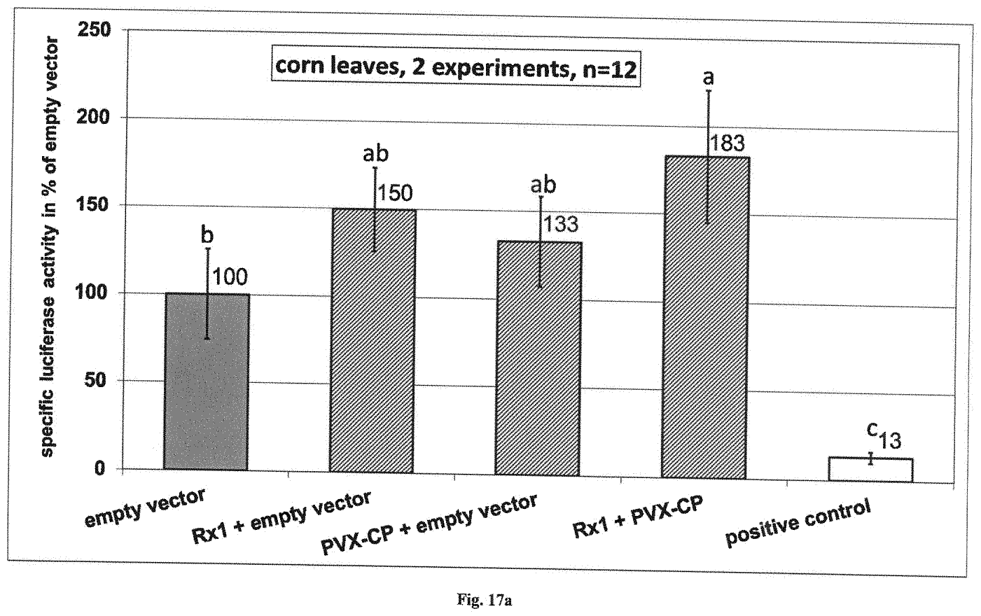

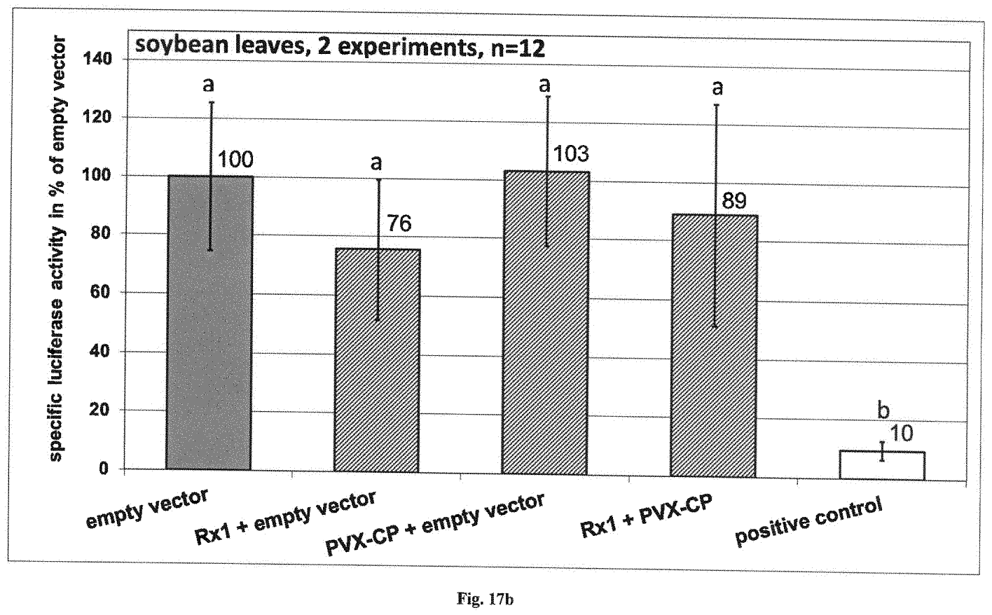

The activity of resistance genes is confined to the members of a plant family, a phenomenon called “restricted taxonomic functionality”. In transient assays, the R3a-Avr3a^{KI} gene combination was tested for cell-death inducing activity in other non-solanaceous crops. After ballistic transformation into leaves of corn, soybean and wheat, a strong induction of cell death was unexpectedly detected as a hallmark of innate immunity (FIG. 15a, 15b, 15c). Other resistance genes of potato and their matching Avr genes (R1-Avr1, Rx1-potato virus X coat protein) did not induce cell death (FIGS. 16a, 16b, 16c and 17a, 17b and 17c). This means that the developed technology can also be transferred to other crops for the generation of broad pathogen resistance.

### CITED DOCUMENTS

Armstrong, M. R., Whisson, S. C., Pritchard, L., Bos, J. I., Venter, E., Avrova, A. O., . . . & Hamlin, N. (2005). An ancestral oomycete locus contains late blight avirulence gene Avr3a, encoding a protein that is recognized in the host cytoplasm. Proceedings of the National Academy of Sciences of the United States of America, 102(21), 7766-7771.
Ayliffe, M. A., & Lagudah, E. S. (2004). Molecular genetics of disease resistance in cereals. Annals of Botany, 94(6), 765-773.
Bos, J. I., Armstrong, M. R., Gilroy, E. M., Boevink, P. C., Hein, I., Taylor, R. M., . . . & Dixelius, C. (2010). Phytophthora infestans effector AVR3a is essential for virulence and manipulates plant immunity by stabilizing host E3 ligase CMPG1. Proceedings of the National Academy of Sciences, 107(21), 9909-9914.
Bourras, S., McNally, K. E., Ben-David, R., Parlange, F., Roffler, S., Praz, C. R., . . . & Schaefer, L. K. (2015). Multiple avirulence loci and allele-specific effector recognition control the Pm3 race-specific resistance of wheat to powdery mildew. The Plant Cell, 27(10), 2991-3012.
Brutus, A., Sicilia, F., Macone, A., Cervone, F., & De Lorenzo, G. (2010). A domain swap approach reveals a role of the plant wall-associated kinase 1 (WAK1) as a receptor of oligogalacturonides. Proceedings of the National Academy of Sciences, 107(20), 9452-9457.
Césari, S., Kanzaki, H., Fujiwara, T., Bernoux, M., Chalvon, V., Kawano, Y., . . . & Kroj, T. (2014). The NB-LRR proteins RGA4 and RGA5 interact functionally and physically to confer disease resistance. The EMBO journal, 33(17), 1941-1959.
Cesari, S., Moore, J., Chen, C., Webb, D., Periyannan, S., Mago, R., . . . & Dodds, P. N. (2016). Cytosolic activation of cell death and stem rust resistance by cereal MLA-family CC-NLR proteins. Proceedings of the National Academy of Sciences, 113(36), 10204-10209.
Chaparro-Garcia, A., Schwizer, S., Sklenar, J., Yoshida, K., Petre, B., Bos, J. I., . . . & Kamoun, S. (2015). Phytophthora infestans RXLR-WY effector AVR3a associates with dynamin-related protein 2 required for endocytosis of the plant pattern recognition receptor FLS2. PLoS One, 10(9), e0137071.
Collier, S. M., & Moffett, P. (2009). NB-LRRs work a “bait and switch” on pathogens. Trends in plant science, 14(10), 521-529.
Collier, S. M., Hamel, L. P., & Moffett, P. (2011). Cell death mediated by the N-terminal domains of a unique and highly conserved class of NB-LRR protein. Molecular plant-microbe interactions, 24(8), 918-931.
Dangl, J. L., Horvath, D. M., & Staskawicz, B. J. (2013). Pivoting the plant immune system from dissection to deployment. Science, 341(6147), 746-751.
Dangl, J. L., & Jones, J. D. (2001). Plant pathogens and integrated defence responses to infection. nature, 411(6839), 826.
Depicker, A., Stachel, S., Dhaese, P., Zambryski, P., & Goodman, H. M. (1982). Nopaline synthase: transcript mapping and DNA sequence. Journal of molecular and applied genetics, 1(6), 561-573.
Fradin, E. F., Abd-El-Haliem, A., Masini, L., van den Berg, G. C., Joosten, M. H., & Thomma, B. P. (2011). Interfamily transfer of tomato Ve1 mediates Verticillium resistance in Arabidopsis. Plant physiology, 156(4), 2255-2265.
Gaudet, R. G., & Gray-Owen, S. D. (2016). Heptose sounds the alarm: Innate sensing of a bacterial sugar stimulates immunity. PLoS pathogens, 12(9), e1005807.
Huang, S., Van Der Vossen, E. A., Kuang, H., Vleeshouwers, V. G., Zhang, N., Borm, T. J., . . . & Visser, R. G. (2005). Comparative genomics enabled the isolation of the R3a late blight resistance gene in potato. The Plant Journal, 42(2), 251-261.
Jones, J. D., & Dangl, J. L. (2006). The plant immune system. Nature, 444(7117), 323.
Joosten, M. H., & de Wit, P. J. (1999). The Tomato—Cladosporium Fulvum Interaction: A Versatile Experimental System to Study Plant-Pathogen Interactions. Annual review of phytopathology, 37(1), 335-367.
Lindsey, K., & Gallois, P. (1990). Transformation of sugarbeet (Beta vulgaris) by Agrobacterium tumefaciens. Journal of experimental botany, 41(5), 529-536.
Lu, X., Kracher, B., Saur, I. M., Bauer, S., Ellwood, S. R., Wise, R., . . . & Schulze-Lefert, P. (2016). Allelic barley MLA immune receptors recognize sequence-unrelated avirulence effectors of the powdery mildew pathogen. Proceedings of the National Academy of Sciences, 113(42), E6486-E6495.
Macho, A. P., & Zipfel, C. (2014). Plant PRRs and the activation of innate immune signaling. Molecular cell, 54(2), 263-272.
Martini, N., Egen, M., Rintz, I., & Strittmatter, G. (1993). Promoter sequences of a potato pathogenesis-related gene mediate transcriptional activation selectively upon fungal infection. Molecular and General Genetics MGG, 236(2-3), 179-186.
McHale, L., Tan, X., Koehl, P., & Michelmore, R. W. (2006). Plant NBS-LRR proteins: adaptable guards. Genome biology, 7(4), 212.
Narusaka, M., Kubo, Y., Hatakeyama, K., Imamura, J., Ezura, H., Nanasato, Y., . . . & Narusaka, Y. (2013). Interfamily transfer of dual NB-LRR genes confers resistance to multiple pathogens. PLoS One, 8(2), e55954.
Niemeyer, J., Ruhe, J., Machens, F., & Hehl, R. (2013). Differential expression of the TMV resistance gene N prevents a hypersensitive response in seeds and during germination. Planta, 237(3), 909-915.
Odell, J. T., Nagy, F., & Chua, N. H. (1985). Identification of DNA sequences required for activity of the cauliflower mosaic virus 35S promoter. Nature, 313(6005), 810.
Rairdan, G. J., Collier, S. M., Sacco, M. A., Baldwin, T. T., Boettrich, T., & Moffett, P. (2008). The coiled-coil and nucleotide binding domains of the potato Rx disease resistance protein function in pathogen recognition and signaling. The Plant Cell, 20(3), 739-751.
Rushton, P. J., Torres, J. T., Parniske, M., Wernert, P., Hahlbrock, K., & Somssich, I. E. (1996). Interaction of elicitor-induced DNA-binding proteins with elicitor response elements in the promoters of parsley PR1 genes. The EMBO journal, 15(20), 5690-5700.
Sambrook, J., & Russell, D. W. (2001). Molecular cloning: a laboratory manual 3rd edition. Coldspring-Harbour Laboratory Press, UK.
Strittmatter, G., Gheysen, G., Gianinazzi-Pearson, V., Hahn, K., Niebel, A., Rohde, W., & Tacke, E. (1996). Infections with various types of organisms stimulate transcription from a short promoter fragment of the potato gst1 gene. MPMI-Molecular Plant Microbe Interactions, 9(1), 68-73.
Takken, F. L., Albrecht, M., & Tameling, W. I. (2006). Resistance proteins: molecular switches of plant defence. Current opinion in plant biology, 9(4), 383-390.
Warren, G. S. (1992). Plant biotechnology: comprehensive biotechnology. Second supplement/volume editor, Michael W. Fowler & Graham S. Warren; editor-in-chief, Murray Moo-Young.
de Wit, P. J. (1992). Molecular characterization of gene-for-gene systems in plant-fungus interactions and the application of avirulence genes in control of plant pathogens. Annual review of phytopathology, 30(1), 391-418.

1. A nucleic acid molecule for increasing the resistance of a plant or a part thereof towards at least one plant pathogen, wherein the nucleic acid molecule comprises a nucleotide sequence selected from the group consisting of:
(a) a nucleotide sequence encoding
i) a plant resistance protein of the resistance protein class CC-NBS-LRR and
ii) an avirulence protein;
(b) a nucleotide sequence encoding a plant resistance protein of the resistance protein class CC-NBS-LRR;
(c) a nucleotide sequence encoding an avirulence protein; and
(d) a nucleotide sequence hybridizing under stringent conditions to a sequence which is complementary to a nucleotide sequence according to (a), (b) or (c);
wherein the encoded resistance protein, the encoded avirulence protein, or both, include at least one amino acid substitution wherein the substitution leads to a decrease of cell death during a hypersensitive reaction in comparison to a resistance protein, avirulence protein or both lacking the amino acid substitution.

2. A vector or an expression cassette comprising the nucleic acid molecule according to claim 1.

3. The nucleic acid molecule according to claim 1, wherein a nucleotide sequence encodes
i) a plant resistance protein of the resistance protein class CC-NBS-LRR and
ii) an avirulence protein.

4. The nucleic acid molecule according to claim 1, wherein the nucleic acid molecule encodes a plant resistance protein having at least one amino acid substitution within the CC-domain leading to a decrease of cell death during a hypersensitive reaction in comparison to a resistance protein which lacks the amino acid substitution within the CC-domain.

5. The nucleic acid molecule according to claim 1, wherein the at least one amino acid substitution is a substitution in a plant resistance protein and wherein the substitution is the exchange of
I) a basic, positively charged amino acid against an acidic, negatively charged or an aliphatic amino acid or
II) D or E in a DAE motif against a basic, positively charged amino acid or an aliphatic amino acid.

6. A polypeptide encoded by the nucleic acid molecule according to claim 1.

7. A cell comprising the nucleic acid molecule according to claim 1.

8. A plant or part thereof comprising the cell according to claim 7, or a plant or part thereof regenerated from the cell according to claim 7.

9. A seed or offspring from a plant or part thereof comprising a cell comprising the nucleic acid molecule according to claim 1, or a plant or part thereof regenerated from the cell comprising the nucleic acid molecule according to claim 1.

10. A method for increasing a plant's resistance towards at least one plant pathogen, the method comprising integrating the nucleic acid molecule according to claim 1 into the genome of at least one plant cell and regenerating a plant from that cell.

11. The plant or part thereof according to claim 8 wherein the plant or part thereof belongs to a species which is selected from the group consisting of: Beta vulgaris, Solanum tuberosum, Triticum aestivum, Hordeum vulgare, Secale cereal, Zea mays, Beta vulgaris, Helianthus annuus, Sorghum bicolor, Brassica napus.

12. A method for increasing the yield produced by a plant being cultivated in an area comprising an organism which is pathogenic towards the plant:
a) providing a plant,
b) cultivating the plant in an area comprising an organism which is pathogen towards the plant, and
c) harvesting the plant,
wherein the plant comprises a nucleic acid molecule according to claim 1 for increasing the resistance towards the organism which is pathogenic towards the plant.

13. The method according to claim 12 wherein the organism which is pathogenic towards the plant is selected from the group consisting of: Alternaria solani, Phytophthora infestans, Blumeria graminis, fungi belonging to the genus of Fusarium, Cercospora beticola and Helminthosporium turcicum.

14. The vector or expression cassette according to claim 2, wherein the nucleic acid molecule is operatively linked to a pathogen-inducible promoter.

15. A cell comprising the polypeptide according to claim 6.

16. A cell comprising the vector or expression cassette according to claim 2.

17. A seed or offspring from a plant or part thereof comprising a cell comprising the nucleic acid molecule according to claim 1, wherein the seed or the offspring comprises a polypeptide encoded by the nucleic acid molecule according to claim 1.

18. A seed or offspring from a plant or part thereof comprising a cell comprising the nucleic acid molecule according to claim 1, wherein the seed or the offspring comprises a vector or expression cassette comprising the nucleic acid molecule according to claim 1.

19. A method for increasing a plant's resistance towards at least one plant pathogen, the method comprising transforming a plant cell with a nucleic acid molecule according to claim 1, or a vector or an expression cassette comprising the nucleic acid molecule according to claim 1, and regenerating a plant from that cell.
